(19) United States
(12) Patent Application Publication
(10) Pub. No.: US 2025/0297244 A1
(43) Pub. Date: Sep. 25, 2025

(54) COMPOSITIONS AND METHODS FOR THE DIAGNOSIS AND TREATMENT OF SEVERE COVID 19 AND OTHER INFLAMMATORY AUTOIMMUNE DISORDERS

(71) Applicant: THE CHILDREN'S HOSPITAL OF PHILADELPHIA, Philadelphia, PA (US)

(72) Inventors: Andrew D. WELLS, Berwyn, PA (US); Struan F. A. GRANT, Swarthmore, PA (US); Matthew C. PAHL, Secane, PA (US); Neil ROMBERG, Merion Station, PA (US)

(73) Assignee: THE CHILDREN'S HOSPITAL OF PHILADELPHIA, Philadelphia, PA (US)

(21) Appl. No.: 18/863,517

(22) PCT Filed: May 8, 2023
(86) PCT No.: PCT/US2023/066728

§ 371 Date: Nov. 6, 2024

Related U.S. Application Data
(60) Provisional application No. 63/339,280, filed on May 6, 2022.

(51) Int. Cl. C12N 15/10; G01N 33/50; G16B 35/00

(52) U.S. Cl. CPC C12N 15/1072; C12N 15/1089; G01N 33/5091; G16B 35/00

(57) ABSTRACT

Compositions and methods for the diagnosis and treatment of severe Covid 19 and other inflammatory autoimmune disorders are disclosed.

### CROSS REFERENCE TO RELATED APPLICATION

This application claims priority to U.S. Provisional Patent Application No. 63/339,280, filed on May 6, 2022, which is incorporated herein by reference in its entirety.

### GRANT STATEMENT

This invention was made with government support under grant numbers R01DK122586 and R01AI146026 awarded by the National Institutes of Health. The government has certain rights in the invention.

### FIELD OF THE INVENTION

The present invention relates the fields of viral inflammatory disease and gene mapping. More specifically, the present invention provides compositions and methods for employing new gene targets associated with viral inflammatory diseases such as Covid, particularly severe Covid, and agents targeting these genes for treatment and management of such diseases.

### BACKGROUND OF THE INVENTION

Several publications and patent documents are cited throughout the specification in order to describe the state of the art to which this invention pertains. Each of these citations is incorporated by reference herein as though set forth in full.

SARS-CoV-2 induces a strong immune response dominated by CD4+ and CD8+ T cells reactive to spike antigen-derived epitopes^{1,2} and accompanied by elevated lymphokines and reduced frequencies of T and B cells in the blood. Pan-lymphopenia and higher cytokine levels are associated with severe disease³⁻⁹, and milder disease is associated with higher frequencies of circulating SARS-CoV-2-specific CD4+ and CD8+ T cells^{2,10-13}. The lungs of COVID-19 patients are also enriched for T cells, and SARS-CoV-2-infected monocyte-derived alveolar macrophages and neutrophils producing T cell chemokines are more abundant in patients with severe disease^{10,14}. During anti-viral immune responses, CD4+ T follicular helper cells (TFH) migrate into germinal centers (GC) to help GC B cells differentiate into high affinity antibody-producing plasmablasts¹⁵. Circulating SARS-CoV-2-specific TFH, plasmablasts, and high-affinity Ab are detected in COVID-19 patients, and the frequency of activated TFH and plasmablasts in the blood are associated with neutralizing IgG levels^{11,16-20}. SARS-CoV-2 infection in macaques induces a similar cellular dynamic in the spleen^{21,22}, and the frequency of circulating plasmablasts, naive CD4+ T cells and TFH in humans is associated with disease severity^{6,11,17-19,23}. The immune dynamics of SARS-CoV-2 infection suggests that genetically-encoded factors regulating the differentiation and function of CD4+ T cells, TFH, and germinal center B cells (GCB) likely influence the severity of COVID-19 disease. Recent genome-wide association studies (GWAS) for critically ill COVID-19 patients have revealed a number of loci associated with the trait^{24,25}. However, GWAS does not identify causal effector genes at non-coding signals, and these loci are often presumptively named after the nearest gene.

### SUMMARY OF THE INVENTION

In accordance with the present invention, a method for alleviating severe Covid 19 or other inflammatory disease symptoms in a patient in need thereof is provided. An exemplary method comprises identifying in a nucleic acid containing biological sample, at least one gene shown in FIG. 4B, or variant thereof, which is indicative of the presence of, or altered risk for, severe Covid 19 or other inflammatory disease; and treating said patient with an effective amount of at least one agent which targets said gene harboring said causal variant, thereby alleviating Covid 19 inflammatory disease symptoms. Other inflammatory diseases to be treated include, for example, inflammatory bowel disease, myasthenia gravis, ulcerative colitis, type I diabetes, lupus, celiac disease, allergy, eczema, autoimmune encephalitis, and rheumatoid arthritis. In some embodiments, the sentinel and proxy SNPs implicating GWAS causal variants and genes identified through 3D epigenomics assays are provided in FIG. 4B. Suitable therapeutic agents useful in the practice of the invention are listed in Table 4. In particularly preferred embodiments, said gene is GART and the agent is a GART agonist (e.g., HF, fGAR and recombinantly produced GART). In other embodiments, the gene is selected from one or more of GART, OAS1, OAS2, OAS3, C21orf49, PaXPB1, SON, IL10RB, IFNAR1, INFAR2, DNAJC28, TNFAIBPL1 AP000295.9, FEM1A, DPP9, and DLX3.

Also provided is a method for identifying an agent useful for the treatment of severe Covid 19 or other inflammatory disease comprising incubating i) a cell harboring at least one gene comprising an informative SNP for severe Covid 19 or other inflammatory disease in a cell type of interest and ii) a cell which lacks said informative SNP in the presence and absence of an agent which modulates the function or expression of at least one gene target associated with one or more of severe Covid 19 symptoms and/or other inflammatory disease symptoms; and identifying agents which alter one or more of the inflammation modulating functions of said gene in cells harboring said SNP relative to those lacking said SNP. In certain embodiments of the method, the cells are selected from tonsil follicular T helper cells, naïve CD4+ T cells, naïve CD8+ T cells, memory CD4+ T cells, memory CD8+ T cells, cytotoxic T lymphocytes, naïve B cells, germinal center B cells, Th1 cells, Th2 cells, Th17 cells, NK cells, dendritic cells, monocytes. Suitable gene targets associated with severe Covid 19 or other inflammatory diseases include GART, OAS1, OAS2, OAS3, C21orf49, PaXPB1, SON, IL10RB, IFNAR1, INFAR2, DNAJC28, TNFAIBPL1, AP000295.9, FEM1A, DPP9, and DLX3. Agents targeting some of these molecules are listed in Table 4.

Also disclosed is a method for treatment of severe Covid 19 inflammatory disease comprising administration of an effective amount of a GART agonist, said treatment alleviating Covid 19 symptoms. In certain embodiments, the agent is a GART agonist comprising one or more of THF, fGAR and recombinantly produced GART. In yet another aspect the method can further comprise administration of a steroid. In another approach, the agent can be a GART inhibitor selected from lometrol, pemetrexed and pelitrexol.

In yet another aspect, a method for alleviating symptoms of an inflammatory disease selected from inflammatory bowel disease, ulcerative colitis, Myasthenia Gravis, type I diabetes, lupus, celiac disease, allergy, eczema, autoimmune encephalitis, and rheumatoid arthritis in a patient in need thereof, is provided. An exemplary method entails identifying in a nucleic acid containing biological sample, at least one gene shown in FIG. 4B, or variant thereof, which is indicative of the presence of, or altered risk for said inflammatory disease; and treating said patient with an effective amount of at least one therapeutic agent which targets said gene harboring said causal variant, thereby alleviating inflammatory disease symptoms. The method can also entail administration of a steroid. In certain embodiments, the therapeutic agent is a modulator of a gene shown in FIG. 4B. The therapeutic agent can be a nucleic acid which modulates the expression level of said one or more target genes.

### DESCRIPTION OF THE DRAWINGS

FIGS. 1A-1E. Source of ATAC-seq datasets for hESCs, monocytes, naïve B cells, GCB, naïve CD4+ T cells, and TFH. (FIG. 1A) The number of biological replicates coming from distinct genetic backgrounds and the source publication for each of the datasets used in this study. hESC data was derived from the H09 cell line. (FIG. 1B) Number of open chromatin regions (ENCODE optimal IDR peaks) identified per cell type. (FIG. 1C) Distribution of open chromatin region lengths. (FIG. 1D) Annotation of genomic features overlapping with OCRs. (FIG. 1E) Pairwise comparison of the global open chromatin regions called as OCRs between each cell type. Yellow indicates the overlap (intersection regions (bp)/union of regions (bp) between each cell type comparison).

FIGS. 2A-2E. Source of Capture C data for hESCs, monocytes, naïve B cells, GCB, naïve CD4+ T cells, and TFH. (FIG. 2A) The number of biological replicates coming from distinct genetic backgrounds and the source publication for each of the datasets used in this study. (FIG. 2B) Number regions identified to interact with a promoter. (FIG. 2C) Cumulative distribution of distances between baits and interacting regions. (FIG. 2D) Proportion of promoter interaction that are located within TADs or that cross TADs. (FIG. 2E) The proportion of promoter interactions that were called in bait-to-bait interactions.

FIGS. 3A-3C. Promoter-connected open chromatin is enriched for highly expressed genes and COVID-19 disease risk heritability. (FIG. 3A) The number of OCRs contacting promoters determined by Capture C and those without promoter contacts. (FIG. 3B) Expression measured by transcripts per kilobase million (TPM) of genes with at least one OCR-promoter contact (red) vs. genes without promoter-OCR contacts (blue). Boxplots represent the median expression for each category. Statistical significance was determined using two-sided Wilcoxon rank-sum tests. TPM Range contacted 0-25499.71, non-contacted 177,277.33). Medians: hESC contacting=8.86, non-contacting 0.0621, Monocyte contacting=11.627, non-contacting 0.0123, Naïve B contacting=14.0, non-contacting 0.0289, GCB contacting=14.0, non-contacting 0.0289, Naïve CD4 T contacting=12.8, non-contacting 0.0244 TFH contacting=14.3, non-contacting=0.216) (FIG. 3C) Enrichment of estimated COVID-19 GWAS heritability determined by partitioned score regression for the open chromatin landscape for each cell type. Points indicate calculated enrichment and whiskers indicate 95% confidence interval. The associated FDR for each enrichment is depicted on the right.

FIGS. 4A-4C. Chromosome capture-based variant-to-gene mapping identifies candidate effector genes at COVID-19 GWAS loci. (FIG. 4A) Manhattan plot generated using the summary statistics from the COVID-19 severity GWAS. Genome-wide significant signals are shown together with the number of accessible gene-annotated proxies associated. (FIG. 4B) Depiction of the statistical sentinel-proxy SNP linkages and the PCC-derived physical gene-proxy connections identified in this study. Genes in yellow were implicated by an accessible proxy in the promoter regions, genes in blue were implicated through chromatin-based contact between the promoter region and a distal accessible proxy, and green indicates implication by both promoter and distal proxies. (FIG. 4C) Heatmap depicting genes implicated by variant-to-gene mapping in each cell type in red. Color of each gene corresponds to the signal shown in (FIG. 4A).

FIGS. 5A-5D. UCSC browser tracks depicting chromatin accessibility (grey), promoter interactions (red) and proxy SNPs (black) at each COVID-19 GWAS locus in each cell type.

FIG. 6. Top IPA gene ontology network for genes implicated by COVID-19 V2G.

FIGS. 7A-7B. Immune genes implicated through contact with COVID-19 variants are differentially expressed in patients with SARS-CoV-2 infection and severe COVID-19 disease. Differential gene expression in mild/moderate and severe COVID-19 patients relative to healthy donors quantified by (FIG. 7A) bulk RNA-seq of whole blood leukocytes at various timepoints from Galani et al.³³, and (FIG. 7B) single-cell RNA-seq from peripheral blood from Zhang et al.³⁴ Values represent log₂FC of TPM for each gene relative to the mean of healthy donors, and genes showing disease severity-associated expression are shown in red. Data in (Fog/7B) represent pseudo-bulk RNA-seq of T cells, B cells, or monocytes clustered by single-cell transcript patterns. Genes indicated in red in FIG. 7A and FIG. 7B exhibited severity-associated expression patterns.

FIGS. 8A-8B. In silico prediction of transcription factor binding site disruption by accessible COVID-19 associated proxies. (FIG. 8A) Transcription factors (blue) with binding motifs likely to be disrupted by accessible COVID-19 SNPs and their connected target genes (green). The predicted effect of the SNP on TF binding is indicated in red for decreased affinity and in blue for increased affinity. COVID-19 risk-associated sequence variation at an element connected to IFNAR2 is predicted to increase binding of ZNF410, a zinc finger protein involved in repression of fetal hemoglobin in erythroid cells. Risk variants also increased the predicted affinity of STAT3 for elements at six implicated genes including PAXBP1. Risk variants at PAXBP1 and five other implicated genes were also predicted to reduce binding of the MYC-induced AP4 (TFAP4) oncoprotein and E2A (TCF3), a central transcription factor in lymphocyte development and malignancy. COVID-19 disease variants connected to OAS1 and OAS3 were predicted to affect the binding of 15 distinct transcription factors, including the E proteins TCF3, TCF4, TCF12, and NEUROD2, the RFX family of transcription factors involved in expression of a variety of immune factors including the MHC class II genes, and the plasmablast and TFH factors MIST1 (BHLHA15) and ASCL2. (FIG. 8B) An example of the predicted impact of the COVID-19 risk allele of rs12482556 (red) on binding of TCF3 and TFAP4.

FIG. 9. The top IPA gene ontology network for transcription factors whose binding is predicted to be influenced by COVID-19-associated SNPs.

FIG. 10A-10B. Potential mechanisms by which V2G-implicated genes impact COVID-19 disease severity. (A) SON may control release and processing of SARS-CoV-2 RNA genomes (1), and GART is involved in de novo generation of the purine precursors required for SARS-CoV-2 RNA replication (2). Sensing of dsRNA regulated by OAS1, OAS2, OAS3 and DPP9 may lead to degradation of SARS-CoV-2 genomes and activation of the inflammasome (3,4). SARS-CoV-2-induced activation of NFkB, IRFs and Jun, dampened by SARS-CoV-2-encoded factors (4-M, nsp1, N, PLPro, ORF3b), induces IFNB. Anti-viral signaling is mediated by type I interferon receptors encoded by IFNAR1 and IFNAR2 (5), and the type 111 interferon receptor encoded by IL10RB (6). These processes are known to be inhibited by the SARS-CoV-2-encoded factors nsp1 and ORF6 (7). (B) Sensing of SARS-CoV-2 RNA genomes released upon infection of lung epithelial cells or alveolar macrophages increases expression of components of the antigen processing and presentation machinery, interferons, cytokines, and chemokines (1-3). These processes are regulated by TNFAIP8L1, FEM1A, and DPP9. Type I interferon receptors IFNAR1 and IFNAR2 control T and NK cell function, and IL10RB affects responsiveness to type III interferons and other cytokines (4). The enzymatic activity of GART regulates metabolic and epigenetic processes important for lymphocyte activation, proliferation, and differentiation (5,6).

FIGS. 11A-11C. GART inhibition abrogates germinal center plasmablast output in tonsillar organoids. (A) Day 7 plasmablast frequencies from untreated or lometrexol-treated tonsillar organoids from a representative tonsil donor. (B) Plasmablast frequency diminishment in day 7 lometrexol drugged organoids relative to untreated counterparts from three tonsil donors. *, P≤0.05. (C) B and T cell viability and TFH frequency from these same experiments. Data from each donor are depicted separately.

FIG. 12. Gating strategy for sorting of naïve B cells and GCB cells for this study.

FIG. 13. Dose-dependent inhibition of human CD4+ T cell proliferation by the GART antagonist lometrexol. Human CD4+ T cells were activated in vitro for 72 hours using anti-CD3+anti-CD28 beads, and cell division was monitored by flow cytometry using CFSE, and the number of daughter cells generated was determined.

FIGS. 14A-14B. Dose-dependent effect of lometrexol on IFNg production by murine Th1 cells (FIG. 14A) and IL-4 production by murine Th2 cells (FIG. 14B) following in vitro activation with anti-CD3+anti-CD28 beads for 6 hours.

FIG. 15. Effect of CRISPR-CAS9 targeting of GART on activation-induced IL-2 production by human CD4+ T cells. *P=0.02 FIGS. 16A-16B. Expression of GART in T cells from 10 SLE subjects and 8 healthy control subjects (FIG. 16A, P=0.02, GEO accession GDS4719/212378) and PBMC from 18 RA subjects and 15 healthy control subjects (FIG. 16B, P=0.009, GEO accession GDS3794/ILMN_1793220). In both cases the differential expression is statistically significant.

### DETAILED DESCRIPTION OF THE INVENTION

SARS-CoV-2 infection results in a broad spectrum of COVID-19 disease, from mild or no symptoms to hospitalization and death. COVID-19 disease severity has been associated with some pre-existing conditions and the magnitude of the adaptive immune response to SARS-CoV-2. Recently a genome-wide association study (GWAS) of the risk of critical illness revealed a significant genetic component. To gain insight into how human genetic variation attenuates or exacerbates disease following SARS-CoV-2 infection, we implicated putatively functional COVID risk variants in the cis-regulatory landscapes of human immune cell types with established roles in disease severity and used high-resolution chromatin conformation capture to map these disease-associated elements to their effector genes. This functional genomic approach implicates 16 genes involved in viral replication, the interferon response, and inflammation. Several of these genes (PAXBP1, IFNAR2, OAS1, OAS3, TNFAIP8L1, GART) were differentially expressed in immune cells from patients with severe vs. moderate COVID-19 disease, and we demonstrate a previously unappreciated role for GART in T cell-dependent antibody-producing B cell differentiation in a human tonsillar organoid model. These results provide immunogenetic insight into the basis of COVID-19 disease severity and provide new targets for therapeutics that limit SARS-CoV-2 infection and its resultant life-threatening inflammation.

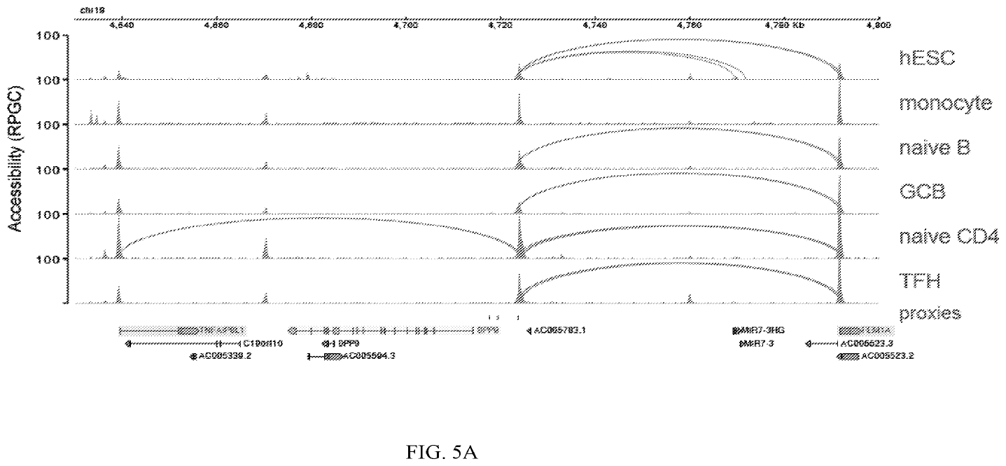

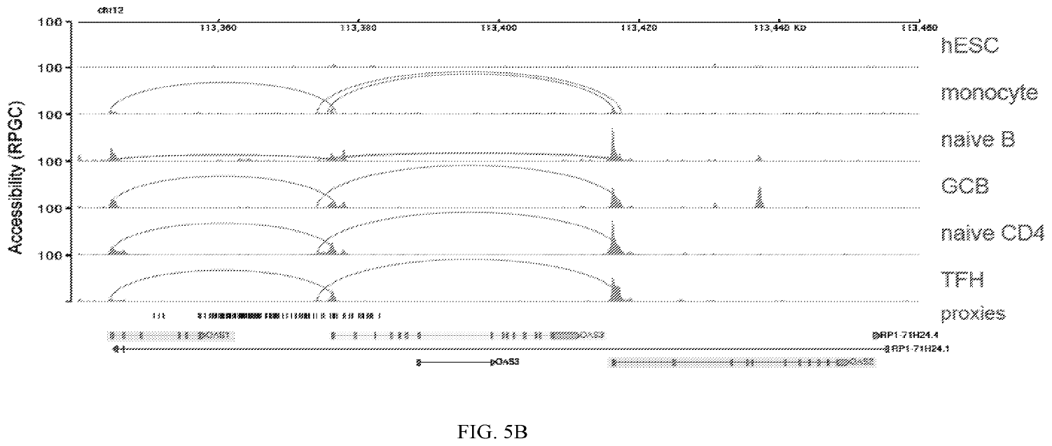

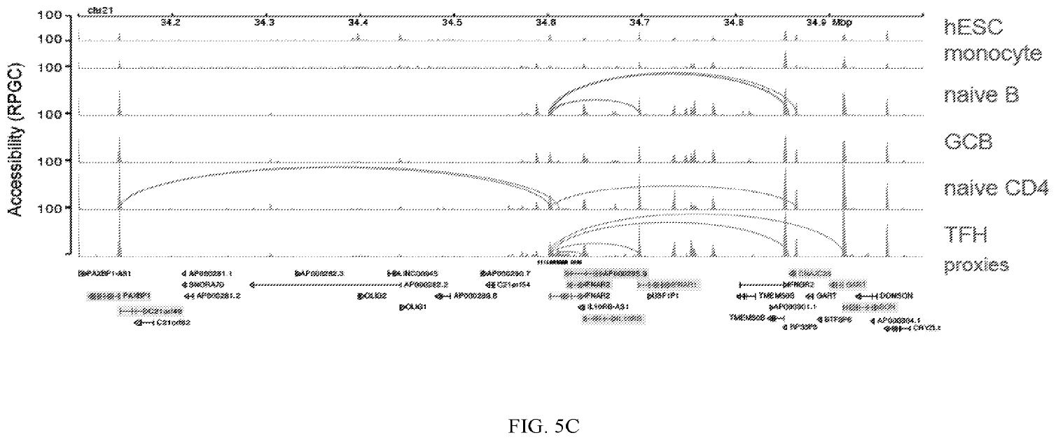

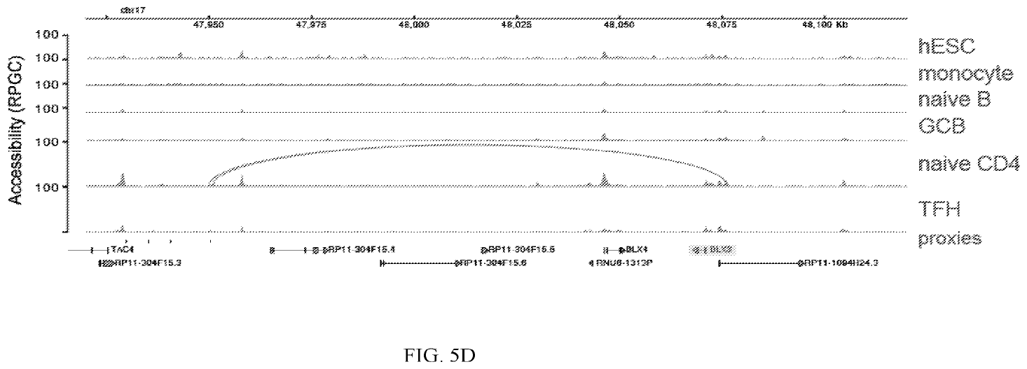

The genes identified through physical association with accessible COVID-19 variants have known roles in viral replication, the interferon response, and inflammation. The genes GART and SON encode factors that may directly impact SARS-CoV-2 replication. SON encodes a factor that regulates HBV influenza A replication, while GART controls de novo purine pools required for coronavirus RNA replication that may also drive evolution of viral variants over the course of the pandemic. Interferons (IFN) are important for the control of early virus replication and in determining moderate vs. severe inflammatory disease. SARS-CoV2 induces type I and type III interferons that signal through IFNAR1, IFNAR2, and IL10RB (FIG. 5A), but SARS-CoV2 also encodes factors that can inhibit type I and III responses. Thus, many SARS-CoV-2-infected individuals exhibit blunted and/or delayed interferon responses, and experience more severe disease than COVID-19 patients with strong interferon responses. SARS-CoV-2 dsRNA genomes are sensed by the RIG-I/MDA5 and RNAseL pathways. OAS1, OAS2, and OAS3 encode crucial regulators of dsRNA degradation by RNAseL, and DPP9 regulates the activity of NLRP1, a dsRNA-sensing component of the inflammasome. Gain of function mutations in OAS1 lead to autoinflammatory disease in humans, polymorphisms at the OAS1 locus are associated with type 2 diabetes⁵⁶, a pre-existing condition associated with severe COVID-19 disease, and genetic variation at DPP9 is associated with the risk of developing pulmonary fibrosis.

Cytokine release syndrome is a major inflammatory complication in patients with severe COVID-19 disease. Receptors for type I (IFNAR1 and 2) and III (IL10RB) interferons drive inflammation mediated by NK and CD8+ T cells, and IL-10RB binds IL-10 whose levels are a severity predictor in COVID-19. FEM1A encodes a negative regulator of NFkB activation, and TNFAIP8L1 regulates expression of the chemokine MCP-1. DNAJC28 is a mitochondrial Hsp40 family member and cofactor of Hsp70 heat shock proteins. PAXBP1 encodes a regulator of ROS and p53, and DLX3 encodes a homeobox protein known to function downstream of the TGFB, BMP, and WNT pathways in tooth and placental development, but immune roles for these factors have not been established. GART encodes an enzyme involved in purine biosynthesis, and its folate-derived metabolites have roles in DNA methylation and mitochondrial redox, processes that regulate immune cell function.

To test for a functional role for GART in adaptive immune responses associated with susceptibility to severe COVID-19, the GART inhibitory drug lometrexol was used in an in vitro human tonsillar organoid model of T cell-dependent germinal center B cell differentiation. After 7 days in culture, T-B interactions in control organoids supported the differentiation of CD27+CD38+ GCB cell plasmablasts capable of producing high-affinity class-switched antibodies in this model. The GART inhibitor lometrexol abrogated plasmablast differentiation in a dose-dependent manner without affecting B or T cell survival or TFH frequency. These results indicate that GART has a previously unappreciated role in T cell-B cell germinal center reactions, and further link GART to immune processes associated with COVID-19 disease severity.

This role for GART in T cell-B cell collaboration for antibody production is completely novel. T cell-B cell collaboration for antibody production is a process involved in immunity against most viral, bacterial, and fungal pathogens, and the efficacy of almost all vaccines against viral, bacterial, and fungal pathogens, therefore targeting GART has utility well beyond COVID-19. For example, therapeutics that promote GART function could act as adjuvants to improve antibody responses elicited from vaccines against COVID-19 (and all associated variants) or other pathogens. Similarly, therapeutics that promote GART function could be used to treat people infected with COVID-19 (and all associated variants) and other pathogens to help them resolve their infections more quickly. Importantly, T cell-B cell collaboration for antibody production is also involved in the generation of disease-causing auto-antibodies in autoimmune disorders such as Lupus, Myasthenia Gravis, Type 1 diabetes, Autoimmune Encephalitis, etc. In this case therapeutics that inhibit GART function could reduce pathogen autoantibody levels and ameliorate disease.

### Definitions

As used herein the term “SARS-CoV-2”, refers to a virus that causes a respiratory disease called coronavirus disease 19 (COVID-19). SARS-CoV-2 is a member of a large family of viruses called coronaviruses. These viruses can infect people and some animals. SARS-CoV-2 was first known to infect people in 2019. The virus is thought to spread from person to person through droplets released when an infected person coughs, sneezes, or talks. It may also be spread by touching a surface with the virus on it and then touching one's mouth, nose, or eyes, but this is less common. SARS-CoV-2 is also called severe acute respiratory syndrome coronavirus 2.

The most common initial symptoms of coronavirus disease 2019 (Covid-19) are cough, fever, fatigue, headache, myalgias, and diarrhea. Severe illness usually begins approximately 1 week after the onset of symptoms. Dyspnea is the most common symptom of severe disease and is often accompanied by hypoxemia. Progressive respiratory failure develops in many patients with severe Covid-19 soon after the onset of dyspnea and hypoxemia. These patients commonly meet the criteria for the acute respiratory distress syndrome (ARDS), which is defined as the acute onset of bilateral infiltrates, severe hypoxemia, and lung edema that is not fully explained by cardiac failure or fluid overload. The majority of patients with severe Covid-19 have lymphopenia, and some have thromboembolic complications as well as disorders of the central or peripheral nervous system. Severe Covid-19 may also lead to acute cardiac, kidney, and liver injury, in addition to cardiac arrhythmias, rhabdomyolysis, coagulopathy, and shock. These organ failures may be associated with clinical and laboratory signs of inflammation, including high fevers, thrombocytopenia, hyperferritinemia, and elevations in C-reactive protein and interleukin-6.

An “autoimmune disease” is a condition in which the body's immune system mistakes its own healthy tissues as foreign and attacks them. Most autoimmune diseases cause inflammation that can affect many parts of the body. The parts of the body affected depend on which autoimmune disease a person has. Common signs and symptoms include fatigue, fever, muscle aches, joint pain and swelling, skin problems, abdominal pain, digestion problems, and swollen glands. The symptoms often come and go and can be mild or severe. There are many different types of autoimmune diseases. They are more common in women and can run in families. Also called autoimmune condition. Compositions are disclosed herein which are useful for the preparation of a medicinal product for treating autoimmune and/or inflammatory diseases due to aberrant levels of autoantibody production in a human subject, and to a method for treating associated with autoimmune and/or inflammatory diseases comprising the administration of the same to a human subject.

The term “diagnosis” refers to a relative probability that a disease (e.g. Covid 19, severe Covid 19, an autoimmune, inflammatory disorder or other disease) is present in the subject. The term “prognosis” refers to a relative probability that a certain future outcome may occur in the subject with respect to a disease state. For example, in the present context, prognosis can refer to the likelihood that an individual will develop Covid 19, or the likely severity of the disease (e.g., extent of pathological effect and duration of disease). The terms are not intended to be absolute, as will be appreciated by any one of skill in the field of medical diagnostics.

As used herein, the term “treatment” or “treating” encompasses prophylaxis and/or therapy. Accordingly, the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Generally, an “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound. As used herein, the term “pharmaceutically effective amount” refers to a dose or quantity that causes improvement in at least one objective or subjective inflammation associated symptom, but not limited to: a reduction in flare ups, joint stiffness, a reduction in neurological symptoms, reduction in or lessening of skin lesion formation, and improvement in kidney function.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, skin cells, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods disclosed herein. The biopsy technique applied will depend on the tissue type to be evaluated (i.e., lung, lymph node, liver, bone marrow, blood cell, joint tissue, synovial tissue, immune cells, hematopoietic cells, fibroblasts, macrophages, T cells, B cells etc.). Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

The phrase “Capture C” refers to a method for profiling chromosomal interactions involving targeted regions of interest, such as gene promoters, globally and at high resolution.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome. Thousands of SNP containing sequences are cataloged on the NCBI SNP database.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

Gene targets newly identified in the methods of the present invention as playing a role in pathologic inflammation include, for example:

“GART” is an intracellular enzyme that controls the synthesis and supply of purines required for coronavirus RNA replication, therefore GART antagonism could be beneficial in treating severe COVID-19 disease by inhibiting SARS-CoV2 replication. The greatest benefit would likely be seen early after infection, before the development of significant inflammation at sites of infection. We also find that inhibition of GART inhibits T cell proliferation, T cell cytokine production, and the differentiation of B cells into effective antibody producers. These anti-inflammatory effects of GART antagonism could be beneficial in treating severe COVID-19-induced inflammation at later stages of the infection, and could benefit systemic autoimmune diseases like myasthenia gravis, rheumatoid and psoriatic arthritis, and lupus. Because GART activity appears to be required for the differentiation of high-affinity antibody-producing B cells in germinal centers, GART agonism could be used as an effective adjuvant in healthy individuals to enhance T cell-dependent antibody responses to vaccination.

“SON” is an intracellular factor known to regulate the replication of two RNA viruses, hepatitis B and influenza A. Presuming that SON is required for SARS-CoV2 replication, antagonism of SON could be beneficial in treating severe COVID-19 disease by inhibiting SARS-CoV2 replication.

SARS-CoV2 induces type I and type III interferons that signal through “IFNAR1”, “IFNAR2”, and “IL10RB”, and are important for the control of early virus replication in many diseases. Many SARS-CoV-2-infected individuals exhibit blunted and/or delayed interferon responses and experience more severe disease than COVID-19 patients with strong interferon responses. Therefore, agonism of “IFNAR1”, “IFNAR2”, and “IL10RB” signaling pathways could limit SARS-CoV2 infection and be beneficial in treating severe COVID-19 disease. The greatest benefit would likely be seen early after infection, before the development of significant inflammation at sites of infection. Early interferon therapy has shown benefits in COVID-19 patients. Targeting of “IFNAR” has been trialed in autoimmune diseases like SLE with varied efficacy and exacerbation of symptoms in some cases.

“OAS1”, “OAS2”, “OAS3”, and “DPP9” are intracellular factors that regulate the sensing and degradation of dsRNA viral genomes by RNAseL and NLRP1, components of the innate immune inflammasome. Gain of function mutations in “OAS1” in humans lead to autoinflammatory disease, and genetic variation at “DPP9” is associated with the risk of developing pulmonary fibrosis. Agonism of these factors and the associated pathway should be beneficial in treating severe COVID-19 disease by inhibiting SARS-CoV2 replication. The greatest benefit would likely be seen early after infection.

“FEM1A” encodes an intracellular negative regulator of NFkB activation, and TNFAIP8L1 is an intracellular factor that negatively regulates production of the chemokine MCP-1. Agonism of these factors or their pathways could be beneficial in treating severe COVID-19-induced inflammation at later stages of the infection and be effective to reduce inflammation in autoimmune diseases.

“PAXBP1” is an intracellular factory that negatively regulates mTORC signaling and the generation of reactive oxygen species, which generally lead to inflammation. Agonism of PAXBP1 could be beneficial in treating severe COVID-19-induced inflammation at later stages of the infection and could also provide therapeutic benefit to patients having systemic autoimmune diseases.

“DLX3” is an intracellular factor that encodes a homeobox protein known to function downstream of the TGF-β, BMP, and WNT pathways. Because in general these are anti-inflammatory pathways, Agonism of “DLX3” should be anti-inflammatory and therefore could provide therapeutic benefits when treating severe COVID-19-induced inflammation at later stages of the infection, and should alleviate symptoms of systemic autoimmune diseases.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In embodiments, antibodies or fragments of antibodies may be derived from different organisms, including humans, mice, rats, hamsters, camels, etc. Antibodies disclosed herein may include antibodies that have been modified or mutated at one or more amino acid positions to improve or modulate a desired function of the antibody (e.g. glycosylation, expression, antigen recognition, effector functions, antigen binding, specificity, etc.).

An “inhibitory nucleic acid” is a nucleic acid (e.g. DNA, RNA, polymer of nucleotide analogs) that is capable of binding to a target nucleic acid and reducing transcription of the target nucleic acid (e.g. mRNA from DNA) or reducing the translation of the target nucleic acid (e.g., mRNA) or altering transcript splicing (e.g. single stranded morpholino oligo). A “morpholino oligo” may be alternatively referred to as a “morpholino nucleic acid” and refers to morpholine-containing nucleic acid nucleic acids commonly known in the art (e.g. phosphoramidate morpholinio oligo or a “PMO”). See Marcos, P., Biochemical and Biophysical Research Communications 358 (2007) 521-527. In embodiments, the “inhibitory nucleic acid” is a nucleic acid that is capable of binding (e.g. hybridizing) to a target nucleic acid (e.g. an mRNA translatable into a protein) and reducing translation of the target nucleic acid. The target nucleic acid is or includes one or more target nucleic acid sequences to which the inhibitory nucleic acid binds (e.g. hybridizes). Thus, an inhibitory nucleic acid typically is or includes a sequence (also referred to herein as an “antisense nucleic acid sequence”) that is capable of hybridizing to at least a portion of a target nucleic acid at a target nucleic acid sequence. An example of an inhibitory nucleic acid is an antisense nucleic acid.

An “antisense nucleic acid” is a nucleic acid (e.g. DNA, RNA or analogs thereof) that is at least partially complementary to at least a portion of a specific target nucleic acid (e.g. a target nucleic acid sequence), such as an mRNA molecule (e.g. a target mRNA molecule) (see, e.g., Weintraub, Scientific American, 262:40 (1990)), for example antisense, siRNA, shRNA, shmiRNA, miRNA (microRNA). Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and sugar-phosphate, backbone-modified nucleotides. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. In embodiments, the inhibitory nucleic acid is single stranded. In embodiments, the inhibitory nucleic acid is double stranded.

In embodiments, an antisense nucleic acid is a morpholino oligo. In embodiments, a morpholino oligo is a single stranded antisense nucleic acid, as is known in the art. In embodiments, a morpholino oligo decreases protein expression of a target, reduces translation of the target mRNA, reduces translation initiation of the target mRNA, or modifies transcript splicing. In embodiments, the morpholino oligo is conjugated to a cell permeable moiety (e.g. peptide). Antisense nucleic acids may be single or double stranded nucleic acids.

In the cell, the antisense nucleic acids may hybridize to the target mRNA, forming a double-stranded molecule. The antisense nucleic acids, interfere with the translation of the mRNA, since the cell will not translate a mRNA that is double-stranded. The use of antisense methods to inhibit the in vitro translation of genes is well known in the art (Marcus-Sakura, Anal. Biochem., 172:289, (1988)). Antisense molecules which bind directly to the DNA may be used.

The compositions of the invention, including without limitation, small molecules, kinase inhibitors and inhibitory nucleic acids can be delivered to the subject using any appropriate means known in the art, including by injection, inhalation, or oral ingestion. Another suitable delivery system is a colloidal dispersion system such as, for example, macromolecule complexes, nanocapsules, microspheres, beads, and lipid-based systems including oil-in-water emulsions, micelles, mixed micelles, and liposomes. An example of a colloidal system is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art Inhibitory nucleic acids (e.g. antisense nucleic acids, morpholino oligos) may be delivered to a cell using cell permeable delivery systems (e.g. cell permeable peptides). In embodiments, inhibitory nucleic acids are delivered to specific cells or tissues using viral vectors or viruses.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

The siRNA can be administered directly, or siRNA expression vectors can be used to induce RNAi that have different design criteria. A vector can have inserted two inverted repeats separated by a short spacer sequence and ending with a string of T's which serve to terminate transcription.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon. polyacrylamide, dextran or agarose.

The phrase “consisting essentially of when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with inflammatory disease. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated.

The term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus, if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any inflammatory disease specific marker gene or nucleic acid but does not hybridize to other nucleotides. Also, polynucleotides which “specifically hybridizes” may hybridize only to an inflammatory disease specific marker, such an inflammatory disease-specific marker shown in the Appendix contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log [Na+]+0.41 (% G+C)−0.63 (% formamide)−600/#bp in duplex

As an illustration of the above formula, using [Na+]=[0.368] and 50% formamide, with GC content of 42% and an average probe size of 200 bases, the T is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. Regarding the nucleic acids of the current invention, a moderate stringency hybridization is defined as hybridization in 6×SSC, 5×Denhardt's solution, 0.5% SDS and 100 μg/ml denatured salmon sperm DNA at 42° C. and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C. and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C. and washing in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension, product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the inflammatory disease specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the inflammatory disease specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the proteins encoded by the inflammatory disease associated nucleic acids described herein. Agents are evaluated for potential biological activity by inclusion in screening assays described hereinbelow.

### Kits and Articles of Manufacture

Any of the aforementioned products can be incorporated into a kit which may contain a Covid 19 inflammatory disease-associated specific marker polynucleotide or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

### Methods of Using Covid 19 Inflammatory Disease-Associated Specific Markers for Development of Therapeutic Agents

Since the genes identified herein have been associated with the etiology of severe Covid 19 and/or inflammatory disease, methods for identifying agents that modulate the activity of the genes and their encoded products the identified SNPs should result in the generation of efficacious therapeutic agents for the treatment of a variety of disorders associated with this condition.

As can be seen from the data provided herein, several chromosomes contain regions which provide suitable targets for the rational design of therapeutic agents which modulate their activity. Small peptide molecules corresponding to these regions may be used to advantage in the design of therapeutic agents which effectively modulate the activity of the encoded proteins.

Molecular modeling should facilitate the identification of specific organic molecules with capacity to bind to the active site of the proteins encoded by the Covid 19 and/or inflammatory disease associated nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered Covid 19 or inflammatory disease associated gene. These host cell lines or cells are defective at the polypeptide level. The host cell lines or cells are grown in the presence of drug compound. The rate of cellular metabolism of the host cells is measured to determine if the compound is capable of regulating the cellular metabolism in the defective cells. Host cells contemplated for use in the present invention include but are not limited to bacterial cells, fungal cells, insect cells, mammalian cells, and plant cells. The inflammatory disease-associated DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter. Picchia promoters such as the alcohol oxidase promoter, and Saccharomyces promoters such as the ga14 inducible promoter and the PGK constitutive promoter, as well as neuronal-specific platelet-derived growth factor promoter (PDGF), the Thy-1 promoter, the hamster and mouse Prion promoter (MoPrP), and the Glial fibrillar acidic protein (GFAP) for the expression of transgenes in glial cells.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the Covid 19 and/or inflammatory disease-associated nucleic acids and proteins of the present invention or functional fragments thereof provide a system in which to screen potential compounds or agents for the ability to modulate the development of inflammatory disease, particularly severe Covid. Thus, in one embodiment, the nucleic acid molecules of the invention may be used to create recombinant cell lines for use in assays to identify agents which modulate aspects of cellular metabolism associated with immune cell signaling associated with inflammatory disease. Also provided herein are methods to screen for compounds capable of modulating the function of proteins encoded by the inflammatory disease associated nucleic acids described herein.

Another approach entails the use of phage display libraries engineered to express fragment of the polypeptides encoded by the inflammatory disease associated nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays. Such compound libraries are commercially available from a number of companies including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated into pharmaceutical compositions and utilized for the treatment of inflammatory disease such as severe Covid 19.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of Covid 19 and/or inflammatory disease associated nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of Covid 19 and/or inflammatory disease-associated nucleic acids enables the production of strains of laboratory mice carrying the Covid 19 inflammatory disease-associated nucleic acids of the invention. Transgenic mice expressing the Covid 19 inflammatory disease-associated nucleic acids of the invention provide a model system in which to examine the role of the protein encoded by the nucleic acid (with or without a sentinel SNP) in the development and progression towards inflammatory disease, including sever Covid 19. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic and regulatory processes associated with aberrant inflammation. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of severe Covid 19 inflammatory disease-associated nucleotide sequences and expression of proteins encoded thereby.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated inflammatory disease-associated genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻⁸. Nonhomologous plasmid-chromosome interactions are more frequent occurring at levels 10⁵-fold to 10²-fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing inflammatory disease-associated SNP containing nucleic acid as a targeted insertional cassette provides means to detect a successful insertion as visualized, for example, by acquisition of immunoreactivity to an antibody immunologically specific for the polypeptide encoded by Covid 19 inflammatory disease-associated nucleic acid and, therefore, facilitates screening/selection of ES cells with the desired genotype.

As used herein, a knock-in animal is one in which the endogenous murine gene, for example, has been replaced with human Covid 19 inflammatory disease-associated gene of the invention. Such knock-in animals provide an ideal model system for studying the development of inflammatory disease.

As used herein, the expression of a Covid 19 inflammatory disease-associated nucleic acid, fragment thereof, or an inflammatory disease-associated fusion protein can be targeted in a “tissue specific manner” or “cell type specific manner” using a vector in which nucleic acid sequences encoding all or a portion of inflammatory disease-associated nucleic acid are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein. The nucleic acid sequence encoding the inflammatory disease-associated sequence of the invention may be operably linked to a variety of different promoter sequences for expression in transgenic animals. Such promoters include, but are not limited to a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter; a neuronal-specific platelet-derived growth factor B gene promoter; FOXP3 promoter for expression specifically in regulatory T cells and Glial fibrillar acidic protein (GFAP) promoter for the expression of transgenes in glial cells.

In certain embodiments, a conditional GART knock out mouse can be constructed to assess the impact of deletion of GART in specific immune cell types on immune responses to foreign and self-antigens. GART knock out mice can also be generated.

Methods of use for the transgenic mice of the invention are also provided herein. Transgenic mice into which a nucleic acid containing the Covid 19 inflammatory disease-associated nucleic acid, or its encoded protein have been introduced are useful, for example, to develop screening methods to screen therapeutic agents to identify those capable of modulating the development of inflammatory disease.

### Pharmaceuticals and Peptide Therapies

The elucidation of the role played by the Covid 19 inflammatory disease associated nucleic acids described herein in inflammation facilitates the development of pharmaceutical compositions useful for treatment and diagnosis of Covid 19 and other inflammatory diseases. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following materials and methods are provided to facilitate the practice of the present invention.

### Antibodies

Anti-human CD19-APC-Cy7 (HIB19, cat #302218), CD27 PerCP-Cy5.5 (0323, cat #302820), and IgD-Pacific-Blue (IA6-2, cat #348225) were from Biolegend. Anti-human CD38-APC (HIT2, cat #555462) and CD21 Pe B-ly4 (557327) were from BD Biosciences.

Purification of B Cells from Human Tonsil

Fresh tonsils were obtained as discarded surgical waste from de-identified immune-competent children undergoing tonsillectomy to address airway obstruction or a history of recurrent tonsillitis. These studies were approved by The Children's Hospital of Philadelphia Institutional Review Board as non-human subject research. The mean age of donors was 5.6 years (range 3-16 years) and 75% were male. Tonsillar mononuclear cells were isolated from tissues by mechanical disruption (tonsils were minced and pressed through a 70-micron cell screen) followed by Ficoll-Paque centrifugation. Naïve B cells (CD19⁺CD21⁺IgD⁺CD38⁻) and GC B cells (CD19⁺CD21⁺CD38⁺IgD⁻CD27⁻) were then sorted using a MoFlo Astrios EQ (Beckman Coulter). The gating strategy is shown in FIG. 12.

### Tonsillar Organoid Preparation and Staining

Excised tonsils from three de-identified immunocompetent patients were diced and strained through a 100 μm filter. A single cell suspension of tonsillar mononuclear cells (MNCs) was created with Ficoll density gradient separation. Once isolated, MNC were counted and resuspended in organoid media (RPMI with L-glutamine, 10% FBS, 2 mM glutamine, 1× penicillin-streptomycin, 1 mM sodium pyruvate, 1×MEM non-essential amino acids, 10 mM HEPES buffer and 1 μg/ml of recombinant human B cell activating factor [BioLegend]) at a concentration of 6×10⁷ cells per ml. As previously described by Wagar et al.⁶⁹, MNC's were transferred to permeable transwells (0.4 m pore, 12 mm diameter; Millipore). Transwells were inserted into standard 12-well polystyrene plates containing 1 ml of additional organoid media and placed in an incubator at 37° C. and 5% CO2. Lometrexol in phosphate buffered saline was added, or not, to organoids after 48 hours in culture. Organoid media with or without lometrexol was replaced every 3 days. On culture day 7, organoid MNCs were resuspended and stained at 4° C. with the anti-human CD38 (HIT2; BioLegend), CD27 (0323; BioLegend), CD19 (HIB19; BioLegend) and L/D aqua (Invitrogen). Cells were analyzed with LSRFortessa (BD Bioscience) and visualized with FlowJo software (TreeStar).

### Cell Fixation

We used standard methods for cell fixation^{26,28-31}. Briefly, 10⁷ naïve or germinal center B cells were suspended in 10 mL RPMI+10% FBS, followed by an additional 270 μL of 37% formaldehyde and incubation for 10 min at RT on a platform rocker. The fixation reaction was quenched by the addition of 1.5 mL cold 1M glycine (4° C.). Fixed cells were centrifuged at 210×g for 5 min at 4° C. and supernatants were removed. The cell pellets were washed in 10 ml cold PBS (4° C.) followed by centrifugation as above. Cell pellets were resuspended in 5 ml cold lysis buffer (10 mM Tris pH8, 10 mM NaCl, 0.2% NP-40/Igepal supplemented with a protease inhibitor cocktail). Resuspended cell pellets were incubated for 20 minutes on ice, centrifuged at 680×g, and lysis buffer was removed. Cell pellets were resuspended in 1 mL of fresh lysis buffer, transferred to 1.5 mL Eppendorf tubes, and snap frozen in ethanol/dry ice or liquid nitrogen. Frozen cell pellets were stored at −80° C. for 3C library generation.

### 3C Library Generation

We used standard methods for generation of 3C libraries^{26,28-31}. For each library, 10⁷ fixed cells were thawed at 37° C., followed by centrifugation at RT for 5 mins at 1845×g. The cell pellet was resuspended in 1 mL of dH₂O supplemented with 5 μL 200× protease inhibitor cocktail, incubated on ice for 10 mins, then centrifuged. Cell pellet was resuspended to a total volume of 650 μL in dH2O. 50 μL of cell suspension was set aside for pre-digestion QC, and the remaining sample was divided into 3 tubes. Both pre-digestion controls and samples underwent a pre-digestion incubation in a Thermomixer (BenchMark) with the addition of 0.3% SDS, 1× NEB DpnII restriction buffer, and dH₂O for 1 hr at 37° C. shaking at 1,000 rpm. A 1.7% solution of Triton X-100 was added to each tube and shaking was continued for another hour. After pre-digestion incubation, 10 μl of DpnII (NEB, 50 U/μL) was added to each sample tube only and continued shaking along with pre-digestion control until the end of the day. An additional 10 μL of DpnII was added to each digestion reaction and digested overnight. The next day, a further 10 μL DpnII was added and continue shaking for another 2-3 hours. 100 p L of each digestion reaction was then removed, pooled into one 1.5 mL tube, and set aside for digestion efficiency QC. The remaining samples were heat inactivated incubated at 1000 rpm in a MultiTherm for 20 min at 65° C. to inactivate the DpnII, and cooled on ice for 20 additional minutes. Digested samples were ligated with 8 μL of T4 DNA ligase (HC ThermoFisher, 30 U/μL) and 1× ligase buffer at 1,000 rpm overnight at 16° C. in a MultiTherm. The next day, an additional 2 μL of T4 DNA ligase was spiked into each sample and incubated for another few hours. The ligated samples were then de-crosslinked overnight at 65° C. with Proteinase K (20 mg/mL, Denville Scientific) along with pre-digestion and digestion control. The following morning, both controls and ligated samples were incubated for 30 min at 37° C. with RNase A (Millipore), followed by phenol/chloroform extraction, ethanol precipitation at −20° C., the 3C libraries were centrifuged at 1000×g for 45 min at 4° C. to pellet the samples. The controls were centrifuged at 1845×g. The pellets were resuspended in 70% ethanol and centrifuged as described above. The pellets of 3C libraries and controls were resuspended in 300 μL and 20 μL dH₂O, respectively, and stored at −20° C. Sample concentrations were measured by Qubit. Digestion and ligation efficiencies were assessed by gel electrophoresis on a 0.9% agarose gel and also by quantitative PCR (SYBR green, Thermo Fisher).

### Promoter-Capture-C Design

Our promoter-Capture-C approach was designed to leverage the four-cutter restriction enzyme DpnII in order to give high resolution restriction fragments of a median of ˜250 bp^{26,28-31}. This approach also allows for scalable resolution through in silico fragment concatenation. Custom capture baits were designed using Agilent SureSelect RNA probes targeting both ends of the DpnIIrestriction fragments containing promoters for coding mRNA, non-coding RNA, antisense RNA, snRNA, miRNA, snoRNA, and lincRNA transcripts (UCSC lincRNA transcripts and sno/miRNA under GRCh37/hg19 assembly) totaling 36,691 RNA baited fragments through the genome. The capture library was re-annotated under gencodeV19 at both 1-fragment and 4-fragment resolution.

### Promoter-Capture-C Assay

Isolated DNA from 3C libraries was quantified using a Qubit fluorometer (Life technologies), and 10 μg of each library was sheared in dH₂O using a QSonica Q800R to an average fragment size of 350 bp^{26,28-31}. QSonica settings used were 60% amplitude, 30 s on, 30 s off, 2 min intervals, for a total of 5 intervals at 4° C. After shearing, DNA was purified using AMPureXP beads (Agencourt). DNA size was assessed on a Bioanalyzer 2100 using a DNA 1000 Chip (Agilent) and DNA concentration was checked via Qubit. SureSelect XT library prep kits (Agilent) were used to repair DNA ends and for adaptor ligation following the manufacturer protocol. Excess adaptors were removed using AMPureXP beads. Size and concentration were checked by Bioanalyzer using a DNA 1000 Chip and by Qubit fluorometer before hybridization. One microgram of adaptor-ligated library was used as input for the SureSelect XT capture kit using manufacturer protocol and our custom-designed 41K promoter Capture-C library. The quantity and quality of the captured library was assessed by Bioanalyzer using a high sensitivity DNA Chip and by Qubit fluorometer. SureSelect XT libraries were then paired-end sequenced on 8 lanes of Illumina Hiseq 4000 platform (100 bp read length).

### ATAC-seq Library Generation

The tonsillar T cells²⁶, monocytes⁷⁰, and B cell subsets were processed in the same manner. A total of 50,000 to 100,000 sorted cells were centrifuged at 550 g for 5 min at 4° C. The cell pellet was washed with cold PBS and resuspended in 50 μL cold lysis buffer (10 mM Tris-HCl, pH 7.4, 10 mM NaCl, 3 mM MgCl₂, 0.1% NP-40/IGEPAL CA-630) and immediately centrifuged at 550 g for 10 min at 4° C. Nuclei were resuspended in the Nextera transposition reaction mix (25 μl 2× TD Buffer, 2.5 μL Nextera Tn5 transposase (Illumina Cat #FC-121-1030), and 22.5 μl nuclease free H₂O) on ice, then incubated for 45 min at 37° C. The tagmented DNA was then purified using the Qiagen MinElute kit eluted with 10.5 μL Elution Buffer (EB). Ten microliters of purified tagmented DNA was PCR amplified using Nextera primers for 12 cycles to generate each library. PCR reaction was subsequently cleaned up using 1.5× AMPureXP beads (Agencourt), and concentrations were measured by Qubit. Libraries were paired-end sequenced on the Illumina HiSeq 4000 platform (100 bp read length).

### ATAC-seq Analysis

ATAC-seq peaks from libraries of tonsillar T cells²⁶, monocytes⁷⁰, and B cell subsets were called using the ENCODE ATAC-seq pipeline (on the world wide web at encodeproject.org/atac-seq/). Briefly, pair-end reads from three biological replicates for each cell type were aligned to hg19 genome using bowtie2, and duplicate reads were removed from the alignment. Narrow peaks were called independently for each replicate using macs2 (-p 0.01 --nomodel --shift -75 --extsize 150 -B --SPMR --keep-dup all --call-summits) and ENCODE blacklist regions (ENCSR636HFF) were removed from peaks in individual replicates. The IDR optimal peak set for each cell type was used to define open chromatin regions in this study.

### Promoter-Focused Capture-C Analysis

Paired-end reads from three biological replicates were pre-processed using the HiCUP pipeline (v0.5.9)⁷¹, with bowtie2 as aligner and hg19 as the reference genome. Significant promoter interactions at 1-DpnII fragment resolution were called using CHiCAGO (v1.1.8)⁷² with default parameters except for binsize set to 2500. Significant interactions at 4-DpnII fragment resolution were also called using CHiCAGO with artificial .baitmap and .rmap files in which DpnII fragments were concatenated in silico into 4 consecutive fragments using default parameters except for removeAdjacent set to False. The significant interactions (CHiCAGO score>5) from both 1-fragment and 4-fragment resolutions were exported in .ibed format and merged into a single file using custom a PERL script to remove redundant interactions and to keep the max CHiCAGO score for each interaction.

### COVID-19 GWAS Data Integration

We curated the lead SNPs from the recent COVID-19 severity GWAS from the COVID-19 Host Genetics Initiative²⁵. The 13 sentinel SNPs represented 8 independent signals. We identified proxies in LD with significant COVID-19 severity loci using LDLinkR with R²>0.8 in EUR ancestry. We next intersected the COVID-19 sentinel and proxy SNPs with the set of OCR annotated to promoter regions (−1500/+500 bp of TSS) and OCR overlapping promoter interacting regions identified by Capture C. Genomic coordinate overlaps were identified using the R package GenomicRanges (ver 1.42) against the human genome reference hg19.

### Partitioned Heritability LD Score Regression Enrichment Analysis

Partitioned heritability LD Score Regression (v1.0.0) was used to identify enrichment of GWAS summary statistics among open accessible regions identified in each cell type. The baseline analysis was performed using LDSCORE data. (See the world wide web at data.broadinstitute.org/alkesgroup/LDSCORE) with LD scores, regression weights, and allele frequencies from 1000G Phase1 and summary statistics from the COVID-19 Host Genetics Initiative²⁵. We generated partitioned LD score regression annotations for each cell type using the coordinates of the all promoter OCR+promoter-interacting OCR. Finally, the cell-type-specific partitioned LD scores were compared to baseline LD scores to measure enrichment in each cell type independently.

### Transcription Factor Motif Analysis

Transcription factor binding site motifs overlapping with proxies implicated in by variant to gene mapping analysis were identified using the R package motifbreakR (v2.0.0)⁷³ using the Jaspar2018 database as our reference set of position weight matrices⁷⁴. Results were filtered to TFs that expressed in the implicated cell type (TPM>1) and were visualized using Cytoscape (v3.8.2)⁷⁵.

### Rna-Seq Library Generation and Analysis

RNA was isolated from ˜1 million of each cell type using Trizol Re-agent (Invitrogen), purified using the Directzol RNA Miniprep Kit (Zymo Research), and depleted of contaminating genomic DNA using DNAse I. Purified RNA was checked for quality on a Bioanlayzer 2100 using the Nano RNA Chip and samples with RIN>7 were used for RNA-seq library preparation. RNA samples were depleted of rRNA using QIAseq Fastselect RNA removal kit (Qiagen). Samples were then processed for the preparation of libraries using the SMARTer Stranded Total RNA Sample Prep Kit (Takara Bio USA) according to manufacturer's instructions. Briefly, the purified first-strand cDNA is amplified into RNA-seq libraries using SeqAmp DNA Polymerase and the Forward and the Reverse PCR Primers from the Illumina Indexing Primer Set HT for Illumina. Quality and quantity of the libraries was assessed using the Agilent 2100 Bioanalyzer system and Qubit fluorometer (Life Technologies). Sequencing of the finalized libraries was performed on the NovaSeq 6000 platform at the CHOP Center for Spatial and Functional Genomics. For analysis, TPM values of bulk RNA-seq data were calculated using the pseudo-aligner Kallisto 0.46.2⁷⁶. Gene level expression was calculated by combining individual TPM values. The comparison of gene expression level between those with promoters with contacts to OCRs or those lacking contacts with OCRs was performed using an unpaired two-sided Wilcoxon rank-sum test, implemented in the R function wilcox.test.

Covid-19 V2G Gene Integration with External RNA-Seq Datasets

We retrieved bulk RNA-seq fastq datasets associated with Galani et a1³³ from the Sequence Read Archive using sra-tools (SRA Toolkit Development Team; see the world wide web at ncbi.github.io/sra-tools/) and plotted the log 2 fold change data reported for severe or mild COVID-19 compared to the mean of 5 healthy donors. Single-end fastq files were retrieved with fastq-dump. TPM values were calculated using kallisto with the following parameters: --single -1 200 -s 20. The log₂ FC for the genes implicated in our immune cell types was calculated for each COVID19 patient compared. We retrieved processed data for pseudo-bulk comparisons between severe and mild COVID-19 relative to healthy donors from Zhang et a1³⁴.

### Ingenuity Pathway Analysis

Ingenuity pathway analysis (IPA, QIAGEN) was used for gene ontology analysis.

### Data Availability

Monocyte and naïve and germinal center B cell raw ATAC seq and Capture C datasets are deposited in GEO with the accession number GSE174658. The naïve CD4+ T cell and TFH datasets are published²⁶ and available at ArrayExpress (see the world wide web at .ebi.ac.uk/arrayexpress/) with accession numbers E-MTAB-6621 (promoter-Capture-C) and E-MTAB-6617 (ATAC-seq).

### Code Availability

Publicly available analysis software and code were used as described in the methods section.

The following examples are provided to illustrate certain embodiments of the invention. They are not intended to limit the invention in any way.

### Example I

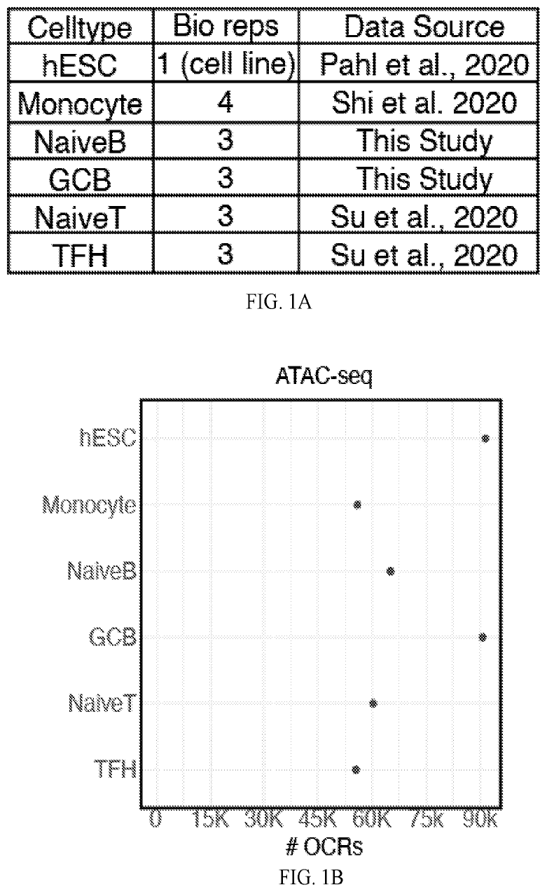

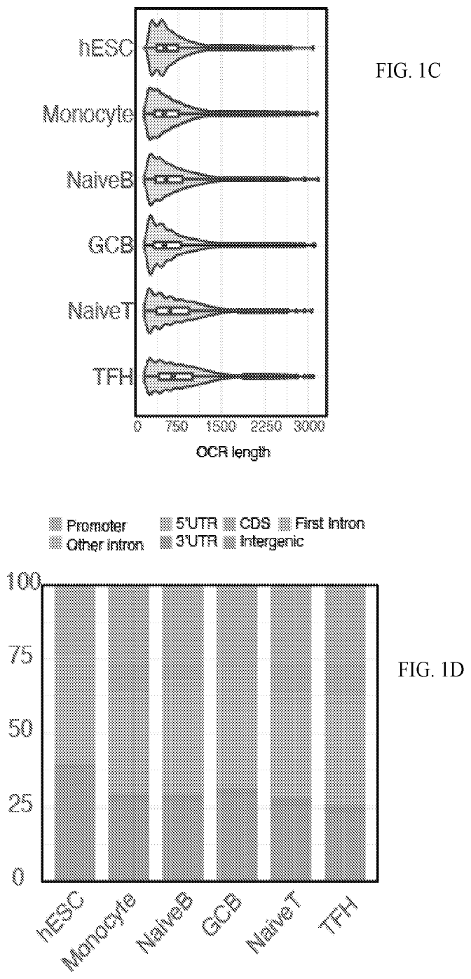

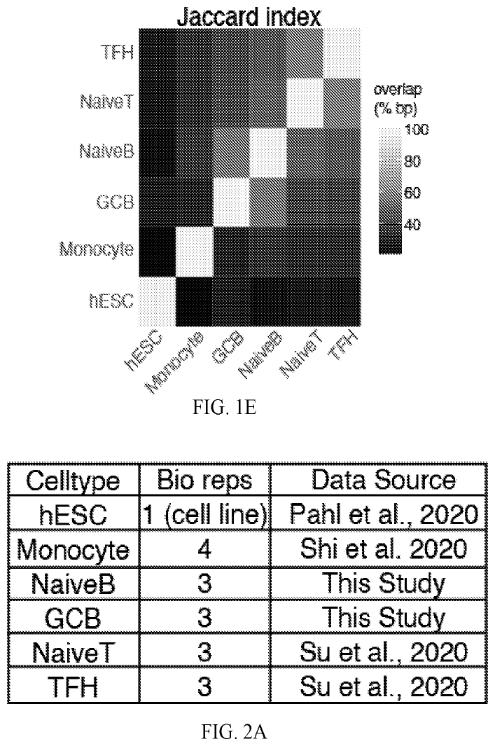

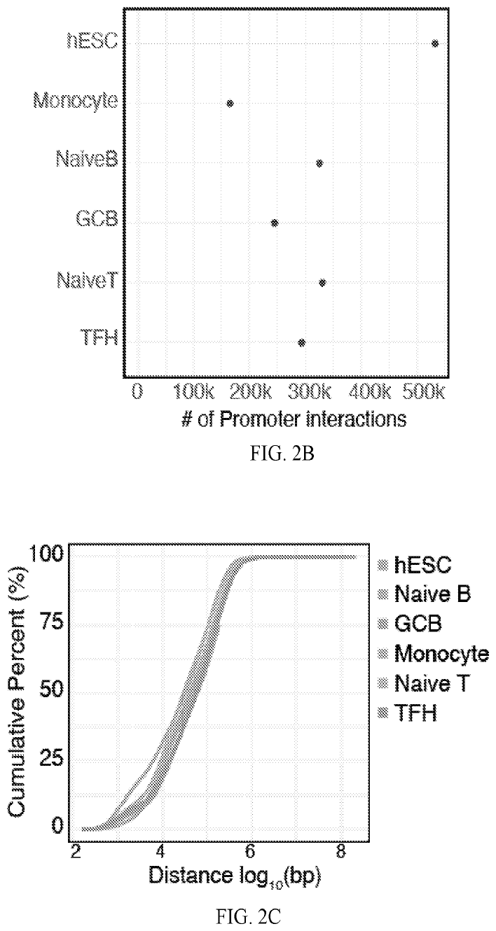

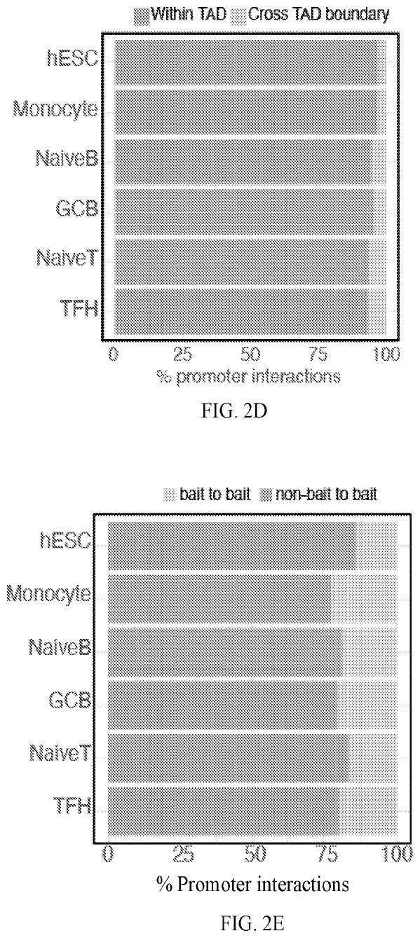

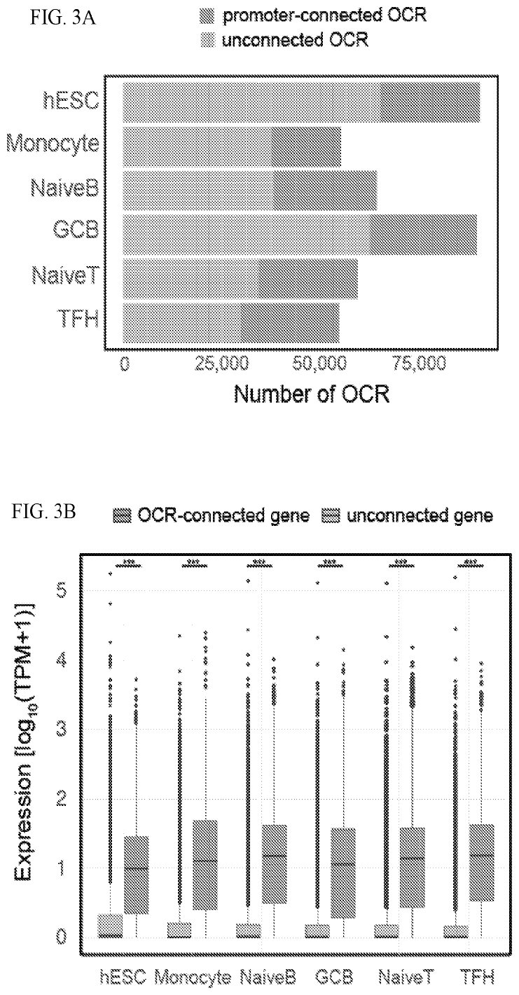

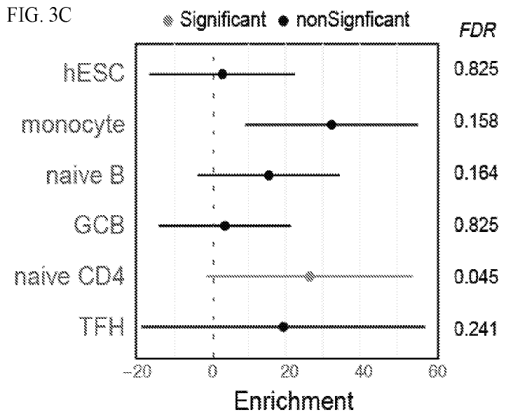

To implicate putative causal variants and their corresponding effector genes at COVID-19 GWAS loci, we leveraged a 3D genomic variant-to-gene mapping approach using disease-relevant, human immune cell types. As an initial step, we used ATAC-seq to identify accessible SNPs in linkage disequilibrium (LD) with GWAS sentinel signals and high-resolution promoter-focused Capture-C (PCC) to connect them to the genes they likely regulate. Because of their connection to COVID-19 disease severity, we chose to perform these analyses in primary naive B cells, naïve CD4+ T cells, follicular helper T cells²⁶ and germinal center B cells from human tonsil, and circulating monocytes (FIG. 1A, Table 1). We included hESC as a non-immune comparator²⁷. The number (range: 55 k-91 k open regions) and genomic distribution (mean range: 496-655 bp) of open chromatin regions (OCR) were comparable among cell types (FIG. 1B-E). To put these OCR in the context of the 3-dimensional structure of the genome, we performed high-resolution PCC targeting the majority of coding and non-coding genes in the human genome^{26,28-31}. The quantity and quality of promoter interactions were similar among immune cell types (FIG. 2, Table 2). One-third to one-half of the open chromatin landscapes were connected to gene promoters (FIG. 3A), and genes whose promoters interact with distal open chromatin regions were expressed ˜10-fold higher than genes not physically associated with open chromatin (FIG. 3B). These results indicate that promoter-connected OCR represent cis-regulatory elements engaged in active control of gene expression. The promoter-connected open chromatin landscape of naive CD4+ T cells was significantly enriched for COVID-19 disease risk heritability (˜28-fold, FDR=0.045, FIG. 3C). TFH and monocyte gene regulatory architectures showed a similar magnitude but more variable enrichment for COVID-19 disease risk variants, and B cells and ESC landscapes did not show enrichment (FIG. 3C). These results are consistent with the growing evidence that dynamics relevant to multiple immune cell types may contribute to disease severity and suggest that COVID-19-associated variants in CD4+ T cell and monocyte open chromatin may have the strongest influence on disease severity.

TABLE 1
ATAC-seq PeakCall Summary

| | Number | | | | | | | | |
|---|---|---|---|---|---|---|---|---|---|
| | IDR | | | | | % OCR | % | % | |
| | optimal | Median | | % OCR | % OCR | gene_{—} | OCR | OCR | |
| | peaks | OCR | % | gene_{—} | gene_{—} | first_{—} | gene_{—} | gene_{—} | % OCR |
| Celltype | (OCRs) | Length | Promoter | 5UTR | CDS | intron | intron | 3UTR | intergenic |
| hESC | 91,528 | 514 | 23.426 | 1.215 | 0.978 | 12.419 | 22.106 | 2.026 | 37.831 |
| Monocyte | 55,902 | 496 | 36.111 | 1.692 | 1.059 | 11.946 | 19.690 | 2.692 | 26.809 |
| NaiveB | 65,113 | 530 | 31.608 | 1.556 | 0.995 | 13.583 | 22.799 | 2.640 | 26.820 |
| GCB | 90,745 | 503 | 26.839 | 1.614 | 1.193 | 14.217 | 24.297 | 2.770 | 29.069 |
| NaiveT | 60,309 | 600 | 35.842 | 1.741 | 1.191 | 12.063 | 20.882 | 3.111 | 25.170 |
| TFH | 55,427 | 655 | 37.272 | 1.817 | 1.227 | 12.739 | 20.943 | 3.067 | 22.935 |

TABLE 2
Summary of Interactions Called by Promoter Capture C

| | | | Percent | | | | Percent |
|---|---|---|---|---|---|---|---|
| | | Median | loops | Percent | | | OCR with |
| | Interaction | Interaction | within | bait to bait | Contacted | Uncontacted | promoter |
| Celltype | Count | Distance | TAD | interactions | OCR | OCR | contact |
| GCB | 245,063 | 50,828 | 95.70% | 20.43% | 27,498 | 63,247 | 30.30% |
| hESC | 532,790 | 32,933 | 97.07% | 14.14% | 25,561 | 65,967 | 27.93% |
| Monocyte | 164,756 | 40,832 | 97.05% | 22.79% | 17,924 | 37,978 | 32.06% |
| NaiveB | 325,381 | 29,612 | 94.59% | 18.96% | 26,455 | 38,658 | 40.63% |
| NaiveT | 330,890 | 59,431 | 93.73% | 16.51% | 25,551 | 34,758 | 42.37% |
| TFH | 293,427 | 56,533 | 93.43% | 19.99% | 25,266 | 30,161 | 45.58% |

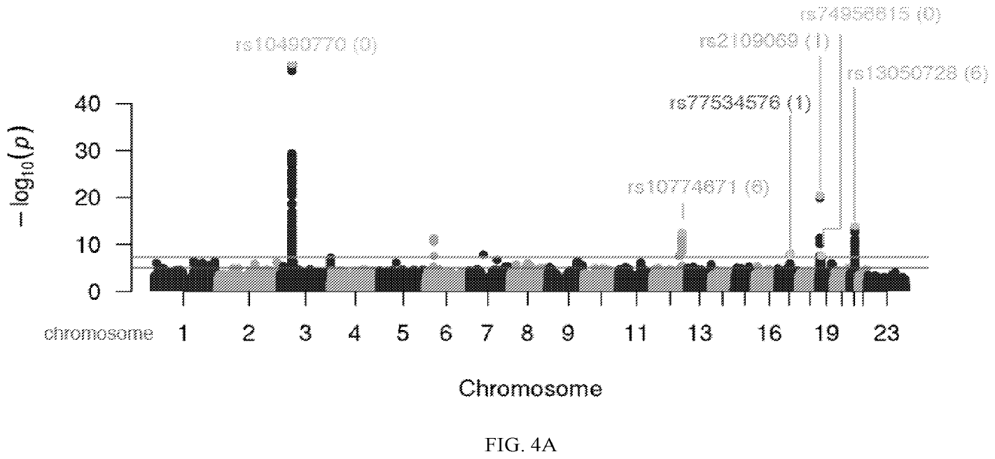

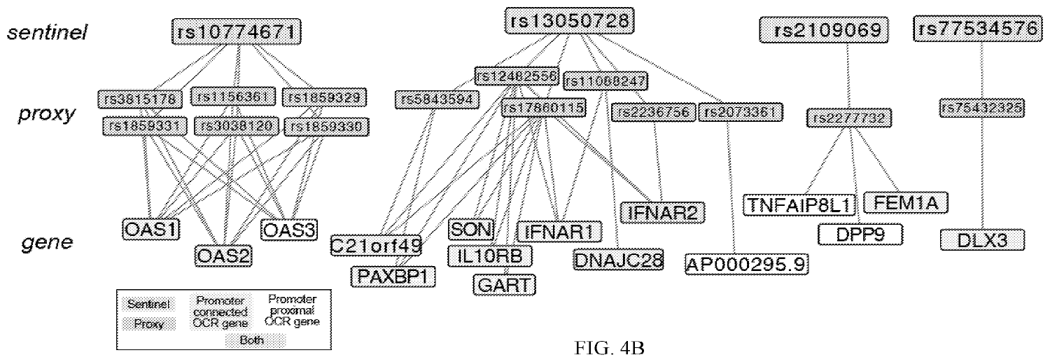

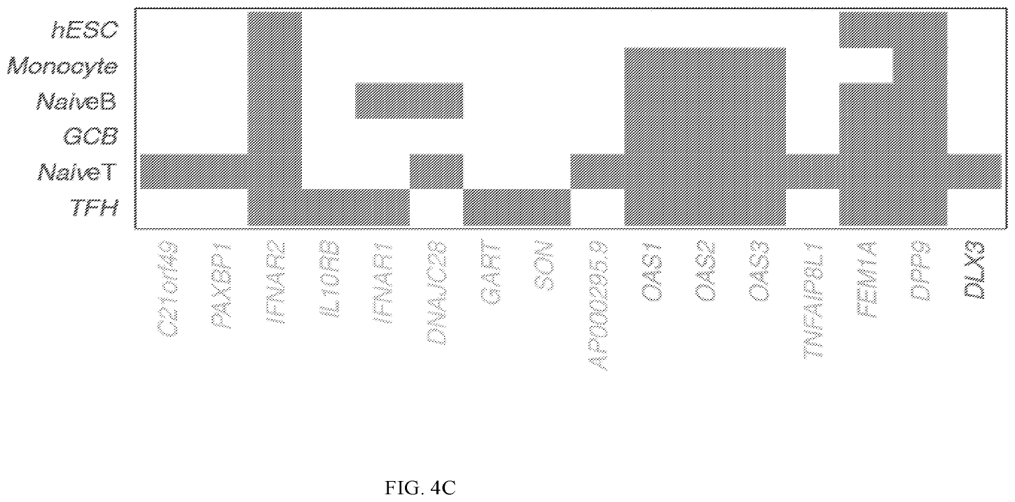

To map potentially functional COVID-19 variants to their target genes, we intersected our ATAC-seq and PCC data with the most recent COVID-19 disease risk GWAS²⁵ (FIGS. 4A and 4B and Table 3). We examined proxy SNPs in high LD (R2>0.8) with the six independent genome-wide significant signals associated with COVID19 severity and identified 16 genes whose promoters physically interact with accessible COVID-19-associated variants in immune cells (FIGS. 4B and 4C, FIG. 5. We identified accessible proxy SNPs in LD with the COVID-19 sentinel rs10774671 in the promoter of OAS3, and accessible proxies interacting with OAS1 and OAS2 in all immune cell types analyzed (FIG. 4C). The DPP9 sentinel rs2109069 is in LD with accessible proxies in the DPP9 promoter in each immune cell type, but also to distal accessible proxies interacting with FEM1A in the T and B cells, and to distal proxies interacting with TNFAIP8L1 specifically in naive CD4+ T cells (FIG. 4C). The sentinel rs13050728 is in LD with accessible proxies interacting with PAXBP1, C21orf49, and AP000295.9 in naive CD4+ T cells, IFNAR1 in TFH and naïve B cells, DNAJC28 in naive T and B cells, GART, IL10RB, and SON in TFH cells, and IFNAR2 in all cell types (FIG. 4C). An proxy SNP in LD with the sentinel rs77534576 is connected to the DLX3 gene in naive CD4+ T cells (FIG. 4C).

TABLE 3
Proxy SNPs in High LD with Six Independent Genome Wide
Significant Signals Associated with Covid-19 Severity

| | | | | | | gene_{—} | |
|---|---|---|---|---|---|---|---|
| Sentinel* | proxy | proxy_pos | R² | cell | proxyOCR | name | gene_id |
| chr21: | rs17860115 | chr21: | 0.9911 | NaiveT | chr21: | C21orf49 | ENSG00000205930.4 |
| 34615210 | | 34602305 | | | 34601880: | | |
| | | | | | 34603506 | | |
| chr21: | rs17860115 | chr21: | 0.9911 | NaiveT | chr21: | PAXBP1 | ENSG00000159086.10 |
| 34615210 | | 34602305 | | | 34601880: | | |
| | | | | | 34603506 | | |
| chr21: | rs12482556 | chr21: | 0.9911 | NaiveT | chr21: | C21orf49 | ENSG00000205930.4 |
| 34615210 | | 34602934 | | | 34601880: | | |
| | | | | | 34603506 | | |
| chr21: | rs12482556 | chr21: | 0.9911 | NaiveT | chr21: | PAXBP1 | ENSG00000159086.10 |
| 34615210 | | 34602934 | | | 34601880: | | |
| | | | | | 34603506 | | |
| chr21: | rs5843594 | chr21: | 0.9866 | NaiveT | chr21: | C21orf49 | ENSG00000205930.4 |
| 34615210 | | 34610985 | | | 34610928: | | |
| | | | | | 34611194 | | |
| chr21: | rs5843594 | chr21: | 0.9866 | NaiveT | chr21: | PAXBP1 | ENSG00000159086.10 |
| 34615210 | | 34610985 | | | 34610928: | | |
| | | | | | 34611194 | | |
| chr21: | rs2236756 | chr21: | 1 | TFH | chr21: | IFNAR2 | ENSG00000159110.15 |
| 34615210 | | 34616923 | | | 34616406: | | |
| | | | | | 34617102 | | |
| chr21: | rs17860115 | chr21: | 0.9911 | TFH | chr21: | IL10RB | ENSG00000243646.4 |
| 34615210 | | 34602305 | | | 34601885: | | |
| | | | | | 34603514 | | |
| chr21: | rs12482556 | chr21: | 0.9911 | TFH | chr21: | IL10RB | ENSG00000243646.4 |
| 34615210 | | 34602934 | | | 34601885: | | |
| | | | | | 34603514 | | |
| chr21: | rs17860115 | chr21: | 0.9911 | NaiveB | chr21: | IFNAR1 | ENSG00000142166.8 |
| 34615210 | | 34602305 | | | 34601888: | | |
| | | | | | 34603502 | | |
| chr21: | rs12482556 | chr21: | 0.9911 | NaiveB | chr21: | IFNAR1 | ENSG00000142166.8 |
| 34615210 | | 34602934 | | | 34601888: | | |
| | | | | | 34603502 | | |
| chr21: | rs11088247 | chr21: | 0.9822 | NaiveB | chr21: | IFNAR1 | ENSG00000142166.8 |
| 34615210 | | 34603249 | | | 34601888: | | |
| | | | | | 34603502 | | |
| chr21: | rs17860115 | chr21: | 0.9911 | TFH | chr21: | IFNAR1 | ENSG00000142166.8 |
| 34615210 | | 34602305 | | | 34601885: | | |
| | | | | | 34603514 | | |
| chr21: | rs12482556 | chr21: | 0.9911 | TFH | chr21: | IFNAR1 | ENSG00000142166.8 |
| 34615210 | | 34602934 | | | 34601885: | | |
| | | | | | 34603514 | | |
| chr21: | rs11088247 | chr21: | 0.9822 | NaiveB | chr21: | DNAJC28 | ENSG00000177692.7 |
| 34615210 | | 34603249 | | | 34601888: | | |
| | | | | | 34603502 | | |
| chr21: | rs11088247 | chr21: | 0.9822 | NaiveT | chr21: | DNAJC28 | ENSG00000177692.7 |
| 34615210 | | 34603249 | | | 34601880: | | |
| | | | | | 34603506 | | |
| chr21: | rs17860115 | chr21: | 0.9911 | TFH | chr21: | GART | ENSG00000159131.12 |
| 34615210 | | 34602305 | | | 34601885: | | |
| | | | | | 34603514 | | |
| chr21: | rs17860115 | chr21: | 0.9911 | TFH | chr21: | SON | ENSG00000159140.13 |
| 34615210 | | 34602305 | | | 34601885: | | |
| | | | | | 34603514 | | |
| chr21: | rs12482556 | chr21: | 0.9911 | TFH | chr21: | GART | ENSG00000159131.12 |
| 34615210 | | 34602934 | | | 34601885: | | |
| | | | | | 34603514 | | |
| chr21: | rs12482556 | chr21: | 0.9911 | TFH | chr21: | SON | ENSG00000159140.13 |
| 34615210 | | 34602934 | | | 34601885: | | |
| | | | | | 34603514 | | |
| chr21: | rs17860115 | chr21: | 0.9911 | GCB | chr21: | IFNAR2 | ENSG00000159110.15 |
| 34615210 | | 34602305 | | | 34601880: | | |
| | | | | | 34603228 | | |
| chr21: | rs12482556 | chr21: | 0.9911 | GCB | chr21: | IFNAR2 | ENSG00000159110.15 |
| 34615210 | | 34602934 | | | 34601880: | | |
| | | | | | 34603228 | | |
| chr21: | rs17860115 | chr21: | 0.9911 | Monocyte | chr21: | IFNAR2 | ENSG00000159110.15 |
| 34615210 | | 34602305 | | | 34601972: | | |
| | | | | | 34603191 | | |
| chr21: | rs12482556 | chr21: | 0.9911 | Monocyte | chr21: | IFNAR2 | ENSG00000159110.15 |
| 34615210 | | 34602934 | | | 34601972: | | |
| | | | | | 34603191 | | |
| chr21: | rs17860115 | chr21: | 0.9911 | NaiveB | chr21: | IFNAR2 | ENSG00000159110.15 |
| 34615210 | | 34602305 | | | 34601888: | | |
| | | | | | 34603502 | | |
| chr21: | rs12482556 | chr21: | 0.9911 | NaiveB | chr21: | IFNAR2 | ENSG00000159110.15 |
| 34615210 | | 34602934 | | | 34601888: | | |
| | | | | | 34603502 | | |
| chr21: | rs17860115 | chr21: | 0.9911 | NaiveT | chr21: | IFNAR2 | ENSG00000159110.15 |
| 34615210 | | 34602305 | | | 34601880: | | |
| | | | | | 34603506 | | |
| chr21: | rs12482556 | chr21: | 0.9911 | NaiveT | chr21: | IFNAR2 | ENSG00000159110.15 |
| 34615210 | | 34602934 | | | 34601880: | | |
| | | | | | 34603506 | | |
| chr21: | rs2073361 | chr21: | 0.9777 | NaiveT | chr21: | AP000295.9 | ENSG00000249624.4 |
| 34615210 | | 34619445 | | | 34619255: | | |
| | | | | | 34619583 | | |
| chr21: | rs17860115 | chr21: | 0.9911 | TFH | chr21: | IFNAR2 | ENSG00000159110.15 |
| 34615210 | | 34602305 | | | 34601885: | | |
| | | | | | 34603514 | | |
| chr21: | rs12482556 | chr21: | 0.9911 | TFH | chr21: | IFNAR2 | ENSG00000159110.15 |
| 34615210 | | 34602934 | | | 34601885: | | |
| | | | | | 34603514 | | |
| chr21: | rs17860115 | chr21: | 0.9911 | hESC | chr21: | IFNAR2 | ENSG00000159110.15 |
| 34615210 | | 34602305 | | | 34601983: | | |
| | | | | | 34603224 | | |
| chr21: | rs12482556 | chr21: | 0.9911 | hESC | chr21: | IFNAR2 | ENSG00000159110.15 |
| 34615210 | | 34602934 | | | 34601983: | | |
| | | | | | 34603224 | | |
| rs10774671 | rs1859331 | chr12: | 0.8925 | GCB | chr12: | OAS1 | ENSG00000089127.8 |
| | | 113376331 | | | 113375662: | | |
| | | | | | 113376733 | | |
| rs10774671 | rs1859330 | chr12: | 0.8925 | GCB | chr12: | OAS1 | ENSG00000089127.8 |
| | | 113376388 | | | 113375662: | | |
| | | | | | 113376733 | | |
| rs10774671 | rs1156361 | chr12: | 0.8797 | GCB | chr12: | OAS1 | ENSG00000089127.8 |
| | | 113375983 | | | 113375662: | | |
| | | | | | 113376733 | | |
| rs10774671 | rs3815178 | chr12: | 0.8712 | GCB | chr12: | OAS1 | ENSG00000089127.8 |
| | | 113376320 | | | 113375662: | | |
| | | | | | 113376733 | | |
| rs10774671 | rs1859329 | chr12: | 0.8712 | GCB | chr12: | OAS1 | ENSG00000089127.8 |
| | | 113376452 | | | 113375662: | | |
| | | | | | 113376733 | | |
| rs10774671 | rs3038120 | chr12: | 0.8673 | GCB | chr12: | OAS1 | ENSG00000089127.8 |
| | | 113376543 | | | 113375662: | | |
| | | | | | 113376733 | | |
| rs10774671 | rs1859331 | chr12: | 0.8925 | Monocyte | chr12: | OAS1 | ENSG00000089127.8 |
| | | 113376331 | | | 113375899: | | |
| | | | | | 113376516 | | |
| rs10774671 | rs1859330 | chr12: | 0.8925 | Monocyte | chr12: | OAS1 | ENSG00000089127.8 |
| | | 113376388 | | | 113375899: | | |
| | | | | | 113376516 | | |
| rs10774671 | rs1156361 | chr12: | 0.8797 | Monocyte | chr12: | OAS1 | ENSG00000089127.8 |
| | | 113375983 | | | 113375899: | | |
| | | | | | 113376516 | | |
| rs10774671 | rs3815178 | chr12: | 0.8712 | Monocyte | chr12: | OAS1 | ENSG00000089127.8 |
| | | 113376320 | | | 113375899: | | |
| | | | | | 113376516 | | |
| rs10774671 | rs1859329 | chr12: | 0.8712 | Monocyte | chr12: | OAS1 | ENSG00000089127.8 |
| | | 113376452 | | | 113375899: | | |
| | | | | | 113376516 | | |
| rs10774671 | rs1859331 | chr12: | 0.8925 | NaiveB | chr12: | OAS1 | ENSG00000089127.8 |
| | | 113376331 | | | 113375922: | | |
| | | | | | 113376556 | | |
| rs10774671 | rs1859330 | chr12: | 0.8925 | NaiveB | chr12: | OAS1 | ENSG00000089127.8 |
| | | 113376388 | | | 113375922: | | |
| | | | | | 113376556 | | |
| rs10774671 | rs1156361 | chr12: | 0.8797 | NaiveB | chr12: | OAS1 | ENSG00000089127.8 |
| | | 113375983 | | | 113375922: | | |
| | | | | | 113376556 | | |
| rs10774671 | rs3815178 | chr12: | 0.8712 | NaiveB | chr12: | OAS1 | ENSG00000089127.8 |
| | | 113376320 | | | 113375922: | | |
| | | | | | 113376556 | | |
| rs10774671 | rs1859329 | chr12: | 0.8712 | NaiveB | chr12: | OAS1 | ENSG00000089127.8 |
| | | 113376452 | | | 113375922: | | |
| | | | | | 113376556 | | |
| rs10774671 | rs3038120 | chr12: | 0.8673 | NaiveB | chr12: | OAS1 | ENSG00000089127.8 |
| | | 113376543 | | | 113375922: | | |
| | | | | | 113376556 | | |
| rs10774671 | rs1859331 | chr12: | 0.8925 | NaiveT | chr12: | OAS1 | ENSG00000089127.8 |
| | | 113376331 | | | 113375663: | | |
| | | | | | 113376727 | | |
| rs10774671 | rs1859330 | chr12: | 0.8925 | NaiveT | chr12: | OAS1 | ENSG00000089127.8 |
| | | 113376388 | | | 113375663: | | |
| | | | | | 113376727 | | |
| rs10774671 | rs1156361 | chr12: | 0.8797 | NaiveT | chr12: | OAS1 | ENSG00000089127.8 |
| | | 113375983 | | | 113375663: | | |
| | | | | | 113376727 | | |
| rs10774671 | rs3815178 | chr12: | 0.8712 | NaiveT | chr12: | OAS1 | ENSG00000089127.8 |
| | | 113376320 | | | 113375663: | | |
| | | | | | 113376727 | | |
| rs10774671 | rs1859329 | chr12: | 0.8712 | NaiveT | chr12: | OAS1 | ENSG00000089127.8 |
| | | 113376452 | | | 113375663: | | |
| | | | | | 113376727 | | |
| rs10774671 | rs3038120 | chr12: | 0.8673 | NaiveT | chr12: | OAS1 | ENSG00000089127.8 |
| | | 113376543 | | | 113375663: | | |
| | | | | | 113376727 | | |
| rs10774671 | rs1859331 | chr12: | 0.8925 | TFH | chr12: | OAS1 | ENSG00000089127.8 |
| | | 113376331 | | | 113375664: | | |
| | | | | | 113376738 | | |
| rs10774671 | rs1859330 | chr12: | 0.8925 | TFH | chr12: | OAS1 | ENSG00000089127.8 |
| | | 113376388 | | | 113375664: | | |
| | | | | | 113376738 | | |
| rs10774671 | rs1156361 | chr12: | 0.8797 | TFH | chr12: | OAS1 | ENSG00000089127.8 |
| | | 113375983 | | | 113375664: | | |
| | | | | | 113376738 | | |
| rs10774671 | rs3815178 | chr12: | 0.8712 | TFH | chr12: | OAS1 | ENSG00000089127.8 |
| | | 113376320 | | | 113375664: | | |
| | | | | | 113376738 | | |
| rs10774671 | rs1859329 | chr12: | 0.8712 | TFH | chr12: | OAS1 | ENSG00000089127.8 |
| | | 113376452 | | | 113375664: | | |
| | | | | | 113376738 | | |
| rs10774671 | rs3038120 | chr12: | 0.8673 | TFH | chr12: | OAS1 | ENSG00000089127.8 |
| | | 113376543 | | | 113375664: | | |
| | | | | | 113376738 | | |
| rs10774671 | rs1859331 | chr12: | 0.8925 | GCB | chr12: | OAS2 | ENSG00000111335.8 |
| | | 113376331 | | | 113375662: | | |
| | | | | | 113376733 | | |
| rs10774671 | rs1859330 | chr12: | 0.8925 | GCB | chr12: | OAS2 | ENSG00000111335.8 |
| | | 113376388 | | | 113375662: | | |
| | | | | | 113376733 | | |
| rs10774671 | rs1156361 | chr12: | 0.8797 | GCB | chr12: | OAS2 | ENSG00000111335.8 |
| | | 113375983 | | | 113375662: | | |
| | | | | | 113376733 | | |
| rs10774671 | rs3815178 | chr12: | 0.8712 | GCB | chr12: | OAS2 | ENSG00000111335.8 |
| | | 113376320 | | | 113375662: | | |
| | | | | | 113376733 | | |
| rs10774671 | rs1859329 | chr12: | 0.8712 | GCB | chr12: | OAS2 | ENSG00000111335.8 |
| | | 113376452 | | | 113375662: | | |
| | | | | | 113376733 | | |
| rs10774671 | rs3038120 | chr12: | 0.8673 | GCB | chr12: | OAS2 | ENSG00000111335.8 |
| | | 113376543 | | | 113375662: | | |
| | | | | | 113376733 | | |
| rs10774671 | rs1859331 | chr12: | 0.8925 | Monocyte | chr12: | OAS2 | ENSG00000111335.8 |
| | | 113376331 | | | 113375899: | | |
| | | | | | 113376516 | | |
| rs10774671 | rs1859330 | chr12: | 0.8925 | Monocyte | chr12: | OAS2 | ENSG00000111335.8 |
| | | 113376388 | | | 113375899: | | |
| | | | | | 113376516 | | |
| rs10774671 | rs1156361 | chr12: | 0.8797 | Monocyte | chr12: | OAS2 | ENSG00000111335.8 |
| | | 113375983 | | | 113375899: | | |
| | | | | | 113376516 | | |
| rs10774671 | rs3815178 | chr12: | 0.8712 | Monocyte | chr12: | OAS2 | ENSG00000111335.8 |
| | | 113376320 | | | 113375899: | | |
| | | | | | 113376516 | | |
| rs10774671 | rs1859329 | chr12: | 0.8712 | Monocyte | chr12: | OAS2 | ENSG00000111335.8 |
| | | 113376452 | | | 113375899: | | |
| | | | | | 113376516 | | |
| rs10774671 | rs1859331 | chr12: | 0.8925 | NaiveB | chr12: | OAS2 | ENSG00000111335.8 |
| | | 113376331 | | | 113375922: | | |
| | | | | | 113376556 | | |
| rs10774671 | rs1859330 | chr12: | 0.8925 | NaiveB | chr12: | OAS2 | ENSG00000111335.8 |
| | | 113376388 | | | 113375922: | | |
| | | | | | 113376556 | | |
| rs10774671 | rs1156361 | chr12: | 0.8797 | NaiveB | chr12: | OAS2 | ENSG00000111335.8 |
| | | 113375983 | | | 113375922: | | |
| | | | | | 113376556 | | |
| rs10774671 | rs3815178 | chr12: | 0.8712 | NaiveB | chr12: | OAS2 | ENSG00000111335.8 |
| | | 113376320 | | | 113375922: | | |
| | | | | | 113376556 | | |
| rs10774671 | rs1859329 | chr12: | 0.8712 | NaiveB | chr12: | OAS2 | ENSG00000111335.8 |
| | | 113376452 | | | 113375922: | | |
| | | | | | 113376556 | | |
| rs10774671 | rs3038120 | chr12: | 0.8673 | NaiveB | chr12: | OAS2 | ENSG00000111335.8 |
| | | 113376543 | | | 113375922: | | |
| | | | | | 113376556 | | |
| rs10774671 | rs1859331 | chr12: | 0.8925 | NaiveT | chr12: | OAS2 | ENSG00000111335.8 |
| | | 113376331 | | | 113375663: | | |
| | | | | | 113376727 | | |
| rs10774671 | rs1859330 | chr12: | 0.8925 | NaiveT | chr12: | OAS2 | ENSG00000111335.8 |
| | | 113376388 | | | 113375663: | | |
| | | | | | 113376727 | | |
| rs10774671 | rs1156361 | chr12: | 0.8797 | NaiveT | chr12: | OAS2 | ENSG00000111335.8 |
| | | 113375983 | | | 113375663: | | |
| | | | | | 113376727 | | |
| rs10774671 | rs3815178 | chr12: | 0.8712 | NaiveT | chr12: | OAS2 | ENSG00000111335.8 |
| | | 113376320 | | | 113375663: | | |
| | | | | | 113376727 | | |
| rs10774671 | rs1859329 | chr12: | 0.8712 | NaiveT | chr12: | OAS2 | ENSG00000111335.8 |
| | | 113376452 | | | 113375663: | | |
| | | | | | 113376727 | | |
| rs10774671 | rs3038120 | chr12: | 0.8673 | NaiveT | chr12: | OAS2 | ENSG00000111335.8 |
| | | 113376543 | | | 113375663: | | |
| | | | | | 113376727 | | |
| rs10774671 | rs1859331 | chr12: | 0.8925 | TFH | chr12: | OAS2 | ENSG00000111335.8 |
| | | 113376331 | | | 113375664: | | |
| | | | | | 113376738 | | |
| rs10774671 | rs1859330 | chr12: | 0.8925 | TFH | chr12: | OAS2 | ENSG00000111335.8 |
| | | 113376388 | | | 113375664: | | |
| | | | | | 113376738 | | |
| rs10774671 | rs1156361 | chr12: | 0.8797 | TFH | chr12: | OAS2 | ENSG00000111335.8 |
| | | 113375983 | | | 113375664: | | |
| | | | | | 113376738 | | |
| rs10774671 | rs3815178 | chr12: | 0.8712 | TFH | chr12: | OAS2 | ENSG00000111335.8 |
| | | 113376320 | | | 113375664: | | |
| | | | | | 113376738 | | |
| rs10774671 | rs1859329 | chr12: | 0.8712 | TFH | chr12: | OAS2 | ENSG00000111335.8 |
| | | 113376452 | | | 113375664: | | |
| | | | | | 113376738 | | |
| rs10774671 | rs3038120 | chr12: | 0.8673 | TFH | chr12: | OAS2 | ENSG00000111335.8 |
| | | 113376543 | | | 113375664: | | |
| | | | | | 113376738 | | |
| rs10774671 | rs1859331 | chr12: | 0.8925 | GCB | chr12: | OAS3 | ENSG00000111331.8 |
| | | 113376331 | | | 113375662: | | |
| | | | | | 113376733 | | |
| rs10774671 | rs1859330 | chr12: | 0.8925 | GCB | chr12: | OAS3 | ENSG00000111331.8 |
| | | 113376388 | | | 113375662: | | |
| | | | | | 113376733 | | |
| rs10774671 | rs1156361 | chr12: | 0.8797 | GCB | chr12: | OAS3 | ENSG00000111331.8 |
| | | 113375983 | | | 113375662: | | |
| | | | | | 113376733 | | |
| rs10774671 | rs3815178 | chr12: | 0.8712 | GCB | chr12: | OAS3 | ENSG00000111331.8 |
| | | 113376320 | | | 113375662: | | |
| | | | | | 113376733 | | |
| rs10774671 | rs1859329 | chr12: | 0.8712 | GCB | chr12: | OAS3 | ENSG00000111331.8 |
| | | 113376452 | | | 113375662: | | |
| | | | | | 113376733 | | |
| rs10774671 | rs3038120 | chr12: | 0.8673 | GCB | chr12: | OAS3 | ENSG00000111331.8 |
| | | 113376543 | | | 113375662: | | |
| | | | | | 113376733 | | |
| rs10774671 | rs1859331 | chr12: | 0.8925 | Monocyte | chr12: | OAS3 | ENSG00000111331.8 |
| | | 113376331 | | | 113375899: | | |
| | | | | | 113376516 | | |
| rs10774671 | rs1859330 | chr12: | 0.8925 | Monocyte | chr12: | OAS3 | ENSG00000111331.8 |
| | | 113376388 | | | 113375899: | | |
| | | | | | 113376516 | | |
| rs10774671 | rs1156361 | chr12: | 0.8797 | Monocyte | chr12: | OAS3 | ENSG00000111331.8 |
| | | 113375983 | | | 113375899: | | |
| | | | | | 113376516 | | |
| rs10774671 | rs3815178 | chr12: | 0.8712 | Monocyte | chr12: | OAS3 | ENSG00000111331.8 |
| | | 113376320 | | | 113375899: | | |
| | | | | | 113376516 | | |
| rs10774671 | rs1859329 | chr12: | 0.8712 | Monocyte | chr12: | OAS3 | ENSG00000111331.8 |
| | | 113376452 | | | 113375899: | | |
| | | | | | 113376516 | | |
| rs10774671 | rs1859331 | chr12: | 0.8925 | NaiveB | chr12: | OAS3 | ENSG00000111331.8 |
| | | 113376331 | | | 113375922: | | |
| | | | | | 113376556 | | |
| rs10774671 | rs1859330 | chr12: | 0.8925 | NaiveB | chr12: | OAS3 | ENSG00000111331.8 |
| | | 113376388 | | | 113375922: | | |
| | | | | | 113376556 | | |
| rs10774671 | rs1156361 | chr12: | 0.8797 | NaiveB | chr12: | OAS3 | ENSG00000111331.8 |
| | | 113375983 | | | 113375922: | | |
| | | | | | 113376556 | | |
| rs10774671 | rs3815178 | chr12: | 0.8712 | NaiveB | chr12: | OAS3 | ENSG00000111331.8 |
| | | 113376320 | | | 113375922: | | |
| | | | | | 113376556 | | |
| rs10774671 | rs1859329 | chr12: | 0.8712 | NaiveB | chr12: | OAS3 | ENSG00000111331.8 |
| | | 113376452 | | | 113375922: | | |
| | | | | | 113376556 | | |
| rs10774671 | rs3038120 | chr12: | 0.8673 | NaiveB | chr12: | OAS3 | ENSG00000111331.8 |
| | | 113376543 | | | 113375922: | | |
| | | | | | 113376556 | | |
| rs10774671 | rs1859331 | chr12: | 0.8925 | NaiveT | chr12: | OAS3 | ENSG00000111331.8 |
| | | 113376331 | | | 113375663: | | |
| | | | | | 113376727 | | |
| rs10774671 | rs1859330 | chr12: | 0.8925 | NaiveT | chr12: | OAS3 | ENSG00000111331.8 |
| | | 113376388 | | | 113375663: | | |
| | | | | | 113376727 | | |
| rs10774671 | rs1156361 | chr12: | 0.8797 | NaiveT | chr12: | OAS3 | ENSG00000111331.8 |
| | | 113375983 | | | 113375663: | | |
| | | | | | 113376727 | | |
| rs10774671 | rs3815178 | chr12: | 0.8712 | NaiveT | chr12: | OAS3 | ENSG00000111331.8 |
| | | 113376320 | | | 113375663: | | |
| | | | | | 113376727 | | |
| rs10774671 | rs1859329 | chr12: | 0.8712 | NaiveT | chr12: | OAS3 | ENSG00000111331.8 |
| | | 113376452 | | | 113375663: | | |
| | | | | | 113376727 | | |
| rs10774671 | rs3038120 | chr12: | 0.8673 | NaiveT | chr12: | OAS3 | ENSG00000111331.8 |
| | | 113376543 | | | 113375663: | | |
| | | | | | 113376727 | | |
| rs10774671 | rs1859331 | chr12: | 0.8925 | TFH | chr12: | OAS3 | ENSG00000111331.8 |
| | | 113376331 | | | 113375664: | | |
| | | | | | 113376738 | | |
| rs10774671 | rs1859330 | chr12: | 0.8925 | TFH | chr12: | OAS3 | ENSG00000111331.8 |
| | | 113376388 | | | 113375664: | | |
| | | | | | 113376738 | | |
| rs10774671 | rs1156361 | chr12: | 0.8797 | TFH | chr12: | OAS3 | ENSG00000111331.8 |
| | | 113375983 | | | 113375664: | | |
| | | | | | 113376738 | | |
| rs10774671 | rs3815178 | chr12: | 0.8712 | TFH | chr12: | OAS3 | ENSG00000111331.8 |
| | | 113376320 | | | 113375664: | | |
| | | | | | 113376738 | | |
| rs10774671 | rs1859329 | chr12: | 0.8712 | TFH | chr12: | OAS3 | ENSG00000111331.8 |
| | | 113376452 | | | 113375664: | | |
| | | | | | 113376738 | | |
| rs10774671 | rs3038120 | chr12: | 0.8673 | TFH | chr12: | OAS3 | ENSG00000111331.8 |
| | | 113376543 | | | 113375664: | | |
| | | | | | 113376738 | | |
| rs2109069 | rs2277732 | chr19: | 0.898 | NaiveT | chr19: | TNFAIP8L1 | ENSG00000185361.4 |
| | | 4723670 | | | 4723485: | | |
| | | | | | 4725078 | | |
| rs2109069 | rs2277732 | chr19: | 0.898 | GCB | chr19: | FEM1A | ENSG00000141965.3 |
| | | 4723670 | | | 4723541: | | |
| | | | | | 4724320 | | |
| rs2109069 | rs2277732 | chr19: | 0.898 | NaiveB | chr19: | FEM1A | ENSG00000141965.3 |
| | | 4723670 | | | 4723439: | | |
| | | | | | 4724867 | | |
| rs2109069 | rs2277732 | chr19: | 0.898 | NaiveT | chr19: | FEM1A | ENSG00000141965.3 |
| | | 4723670 | | | 4723485: | | |
| | | | | | 4725078 | | |
| rs2109069 | rs2277732 | chr19: | 0.898 | TFH | chr19: | FEM1A | ENSG00000141965.3 |
| | | 4723670 | | | 4723427: | | |
| | | | | | 4724449 | | |
| rs2109069 | rs2277732 | chr19: | 0.898 | hESC | chr19: | FEM1A | ENSG00000141965.3 |
| | | 4723670 | | | 4723421: | | |
| | | | | | 4724330 | | |
| rs2109069 | rs2277732 | chr19: | 0.898 | GCB | chr19: | DPP9 | ENSG00000142002.12 |
| | | 4723670 | | | 4723541: | | |
| | | | | | 4724320 | | |
| rs2109069 | rs2277732 | chr19: | 0.898 | Monocyte | chr19: | DPP9 | ENSG00000142002.12 |
| | | 4723670 | | | 4723534: | | |
| | | | | | 4724293 | | |
| rs2109069 | rs2277732 | chr19: | 0.898 | NaiveB | chr19: | DPP9 | ENSG00000142002.12 |
| | | 4723670 | | | 4723439: | | |
| | | | | | 4724867 | | |
| rs2109069 | rs2277732 | chr19: | 0.898 | NaiveT | chr19: | DPP9 | ENSG00000142002.12 |
| | | 4723670 | | | 4723485: | | |
| | | | | | 4725078 | | |
| rs2109069 | rs2277732 | chr19: | 0.898 | TFH | chr19: | DPP9 | ENSG00000142002.12 |
| | | 4723670 | | | 4723427: | | |
| | | | | | 4724449 | | |
| rs2109069 | rs2277732 | chr19: | 0.898 | hESC | chr19: | DPP9 | ENSG00000142002.12 |
| | | 4723670 | | | 4723421: | | |
| | | | | | 4724330 | | |
| rs77534576 | rs75432325 | chr17: | 0.9161 | NaiveT | chr17: | DLX3 | ENSG00000064195.7 |
| | | 47950329 | | | 47950249: | | |
| | | | | | 47951150 | | |

| | | | | open | proxy2 | | | | |
|---|---|---|---|---|---|---|---|---|---|
| Sentinel* | Frag | b2b** | score | Bait | BaitDist | oe_frag | bait_frag | oeGenes | int |
| chr21: | 4frag | T | 8.11 | 1 | 458210 | chr21: | chr21: | IFNAR2 | snpAtOE |
| 34615210 | | | | | | 34600994: | 34142956: | | |
| | | | | | | 34603177 | 34145234 | | |
| chr21: | 4frag | T | 8.11 | 1 | 458210 | chr21: | chr21: | IFNAR2 | snpAtOE |
| 34615210 | | | | | | 34600994: | 34142956: | | |
| | | | | | | 34603177 | 34145234 | | |
| chr21: | 4frag | T | 8.11 | 1 | 458839 | chr21: | chr21: | IFNAR2 | snpAtOE |
| 34615210 | | | | | | 34600994: | 34142956: | | |
| | | | | | | 34603177 | 34145234 | | |
| chr21: | 4frag | T | 8.11 | 1 | 458839 | chr21: | chr21: | IFNAR2 | snpAtOE |
| 34615210 | | | | | | 34600994: | 34142956: | | |
| | | | | | | 34603177 | 34145234 | | |
| chr21: | 4frag | F | 5.68 | 1 | 466890 | chr21: | chr21: | NA | snpAtOE |
| 34615210 | | | | | | 34609199: | 34142956: | | |
| | | | | | | 34612029 | 34145234 | | |
| chr21: | 4frag | F | 5.68 | 1 | 466890 | chr21: | chr21: | NA | snpAtOE |
| 34615210 | | | | | | 34609199: | 34142956: | | |
| | | | | | | 34612029 | 34145234 | | |
| chr21: | 4frag | F | 6.96 | 1 | 14838 | chr21: | chr21: | NA | snpAtOE |
| 34615210 | | | | | | 34616548: | 34600994: | | |
| | | | | | | 34618012 | 34603177 | | |
| chr21: | 4frag | T | 9.53 | 1 | 36365 | chr21: | chr21: | IFNAR2 | snpAtOE |
| 34615210 | | | | | | 34600994: | 34638190: | | |
| | | | | | | 34603177 | 34639150 | | |
| chr21: | 4frag | T | 9.53 | 1 | 35736 | chr21: | chr21: | IFNAR2 | snpAtOE |
| 34615210 | | | | | | 34600994: | 34638190: | | |
| | | | | | | 34603177 | 34639150 | | |
| chr21: | 4frag | T | 9.5 | 1 | 95630 | chr21: | chr21: | IFNAR2 | snpAtOE |
| 34615210 | | | | | | 34600994: | 34696955: | | |
| | | | | | | 34603177 | 34698915 | | |
| chr21: | 4frag | T | 9.5 | 1 | 95001 | chr21: | chr21: | IFNAR2 | snpAtOE |
| 34615210 | | | | | | 34600994: | 34696955: | | |
| | | | | | | 34603177 | 34698915 | | |
| chr21: | 4frag | F | 6.3 | 1 | 94686 | chr21: | chr21: | NA | snpAtOE |
| 34615210 | | | | | | 34603178: | 34696955: | | |
| | | | | | | 34604944 | 34698915 | | |
| chr21: | 4frag | T | 8.68 | 1 | 95630 | chr21: | chr21: | IFNAR2 | snpAtOE |
| 34615210 | | | | | | 34600994: | 34696955: | | |
| | | | | | | 34603177 | 34698915 | | |
| chr21: | 4frag | T | 8.68 | 1 | 95001 | chr21: | chr21: | IFNAR2 | snpAtOE |
| 34615210 | | | | | | 34600994: | 34696955: | | |
| | | | | | | 34603177 | 34698915 | | |
| chr21: | 4frag | F | 5.67 | 1 | 260768 | chr21: | chr21: | NA | snpAtOE |
| 34615210 | | | | | | 34603178: | 34863147: | | |
| | | | | | | 34604944 | 34864887 | | |
| chr21: | 4frag | F | 5.23 | 1 | 260768 | chr21: | chr21: | NA | snpAtOE |
| 34615210 | | | | | | 34603178: | 34863147: | | |
| | | | | | | 34604944 | 34864887 | | |
| chr21: | 4frag | T | 5.49 | 1 | 312554 | chr21: | chr21: | IFNAR2 | snpAtOE |
| 34615210 | | | | | | 34600994: | 34913752: | | |
| | | | | | | 34603177 | 34915966 | | |
| chr21: | 4frag | T | 5.49 | 1 | 312554 | chr21: | chr21: | IFNAR2 | snpAtOE |
| 34615210 | | | | | | 34600994: | 34913752: | | |
| | | | | | | 34603177 | 34915966 | | |
| chr21: | 4frag | T | 5.49 | 1 | 311925 | chr21: | chr21: | IFNAR2 | snpAtOE |
| 34615210 | | | | | | 34600994: | 34913752: | | |
| | | | | | | 34603177 | 34915966 | | |
| chr21: | 4frag | T | 5.49 | 1 | 311925 | chr21: | chr21: | IFNAR2 | snpAtOE |
| 34615210 | | | | | | 34600994: | 34913752: | | |
| | | | | | | 34603177 | 34915966 | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| chr21: | NA | NA | NA | NA | NA | NA | NA | NA | snpAtPromoter |
| 34615210 | | | | | | | | | |
| rs10774671 | 4frag | T | 11.92 | 1 | 31098 | chr12: | chr12: | OAS3 | snpAtOE |
| | | | | | | 113374038: | 113344413: | | |
| | | | | | | 113376755 | 113346052 | | |
| rs10774671 | 4frag | T | 11.92 | 1 | 31156 | chr12: | chr12: | OAS3 | snpAtOE |
| | | | | | | 113374038: | 113344413: | | |
| | | | | | | 113376755 | 113346052 | | |
| rs10774671 | 4frag | T | 11.92 | 1 | 30750 | chr12: | chr12: | OAS3 | snpAtOE |
| | | | | | | 113374038: | 113344413: | | |
| | | | | | | 113376755 | 113346052 | | |
| rs10774671 | 4frag | T | 11.92 | 1 | 31088 | chr12: | chr12: | OAS3 | snpAtOE |
| | | | | | | 113374038: | 113344413: | | |
| | | | | | | 113376755 | 113346052 | | |
| rs10774671 | 4frag | T | 11.92 | 1 | 31220 | chr12: | chr12: | OAS3 | snpAtOE |
| | | | | | | 113374038: | 113344413: | | |
| | | | | | | 113376755 | 113346052 | | |
| rs10774671 | 4frag | T | 11.92 | 1 | 31310 | chr12: | chr12: | OAS3 | snpAtOE |
| | | | | | | 113374038: | 113344413: | | |
| | | | | | | 113376755 | 113346052 | | |
| rs10774671 | 4frag | T | 18.27 | 1 | 31098 | chr12: | chr12: | OAS3 | snpAtOE |
| | | | | | | 113374038: | 113344413: | | |
| | | | | | | 113376755 | 113346052 | | |
| rs10774671 | 4frag | T | 18.27 | 1 | 31156 | chr12: | chr12: | OAS3 | snpAtOE |
| | | | | | | 113374038: | 113344413: | | |
| | | | | | | 113376755 | 113346052 | | |
| rs10774671 | 4frag | T | 18.27 | 1 | 30750 | chr12: | chr12: | OAS3 | snpAtOE |
| | | | | | | 113374038: | 113344413: | | |
| | | | | | | 113376755 | 113346052 | | |
| rs10774671 | 4frag | T | 18.27 | 1 | 31088 | chr12: | chr12: | OAS3 | snpAtOE |
| | | | | | | 113374038: | 113344413: | | |
| | | | | | | 113376755 | 113346052 | | |
| rs10774671 | 4frag | T | 18.27 | 1 | 31220 | chr12: | chr12: | OAS3 | snpAtOE |
| | | | | | | 113374038: | 113344413: | | |
| | | | | | | 113376755 | 113346052 | | |
| rs10774671 | 4frag \| | T | 12.77 | 1 | 31330 | chr12: | chr12: | OAS3 | snpAtOE |
| | 1frag | | | | | 113374038: | 113344413: | | |
| | | | | | | 113376755 \| | 113346052 \| | | |
| | | | | | | chr12: | chr12: | | |
| | | | | | | 113375558: | 113344691: | | |
| | | | | | | 113376755 | 113344850 | | |
| rs10774671 | 4frag \| | T | 12.77 | 1 | 31386 | chr12: | chr12: | OAS3 | snpAtOE |
| | 1frag | | | | | 113374038: | 113344413: | | |
| | | | | | | 113376755 \| | 113346052 \| | | |
| | | | | | | chr12: | chr12: | | |
| | | | | | | 113375558: | 113344691: | | |
| | | | | | | 113376755 | 113344850 | | |
| rs10774671 | 4frag \| | T | 12.77 | 1 | 30982 | chr12: | chr12: | OAS3 | snpAtOE |
| | 1frag | | | | | 113374038: | 113344413: | | |
| | | | | | | 113376755 \| | 113346052 \| | | |
| | | | | | | chr12: | chr12: | | |
| | | | | | | 113375558: | 113344691: | | |
| | | | | | | 113376755 | 113344850 | | |
| rs10774671 | 4frag \| | T | 12.77 | 1 | 31318 | chr12: | chr12: | OAS3 | snpAtOE |
| | 1frag | | | | | 113374038: | 113344413: | | |
| | | | | | | 113376755 \| | 113346052 \| | | |
| | | | | | | chr12: | chr12: | | |
| | | | | | | 113375558: | 113344691: | | |
| | | | | | | 113376755 | 113344850 | | |
| rs10774671 | 4frag \| | T | 12.77 | 1 | 31450 | chr12: | chr12: | OAS3 | snpAtOE |
| | 1frag | | | | | 113374038: | 113344413: | | |
| | | | | | | 113376755 \| | 113346052 \| | | |
| | | | | | | chr12: | chr12: | | |
| | | | | | | 113375558: | 113344691: | | |
| | | | | | | 113376755 | 113344850 | | |
| rs10774671 | 4frag \| | T | 12.77 | 1 | 31542 | chr12: | chr12: | OAS3 | snpAtOE |
| | 1frag | | | | | 113374038: | 113344413: | | |
| | | | | | | 113376755 \| | 113346052 \| | | |
| | | | | | | chr12: | chr12: | | |
| | | | | | | 113375558: | 113344691: | | |
| | | | | | | 113376755 | 113344850 | | |
| rs10774671 | 4frag | T | 15.4 | 1 | 31098 | chr12: | chr12: | OAS3 | snpAtOE |
| | | | | | | 113374038: | 113344413: | | |
| | | | | | | 113376755 | 113346052 | | |
| rs10774671 | 4frag | T | 15.4 | 1 | 31156 | chr12: | chr12: | OAS3 | snpAtOE |
| | | | | | | 113374038: | 113344413: | | |
| | | | | | | 113376755 | 113346052 | | |
| rs10774671 | 4frag | T | 15.4 | 1 | 30750 | chr12: | chr12: | OAS3 | snpAtOE |
| | | | | | | 113374038: | 113344413: | | |
| | | | | | | 113376755 | 113346052 | | |
| rs10774671 | 4frag | T | 15.4 | 1 | 31088 | chr12: | chr12: | OAS3 | snpAtOE |
| | | | | | | 113374038: | 113344413: | | |
| | | | | | | 113376755 | 113346052 | | |
| rs10774671 | 4frag | T | 15.4 | 1 | 31220 | chr12: | chr12: | OAS3 | snpAtOE |
| | | | | | | 113374038: | 113344413: | | |
| | | | | | | 113376755 | 113346052 | | |
| rs10774671 | 4frag | T | 15.4 | 1 | 31310 | chr12: | chr12: | OAS3 | snpAtOE |
| | | | | | | 113374038: | 113344413: | | |
| | | | | | | 113376755 | 113346052 | | |
| rs10774671 | 4frag | T | 7.93 | 1 | 31098 | chr12: | chr12: | OAS3 | snpAtOE |
| | | | | | | 113374038: | 113344413: | | |
| | | | | | | 113376755 | 113346052 | | |
| rs10774671 | 4frag | T | 7.93 | 1 | 31156 | chr12: | chr12: | OAS3 | snpAtOE |
| | | | | | | 113374038: | 113344413: | | |
| | | | | | | 113376755 | 113346052 | | |
| rs10774671 | 4frag | T | 7.93 | 1 | 30750 | chr12: | chr12: | OAS3 | snpAtOE |
| | | | | | | 113374038: | 113344413: | | |
| | | | | | | 113376755 | 113346052 | | |
| rs10774671 | 4frag | T | 7.93 | 1 | 31088 | chr12: | chr12: | OAS3 | snpAtOE |
| | | | | | | 113374038: | 113344413: | | |
| | | | | | | 113376755 | 113346052 | | |
| rs10774671 | 4frag | T | 7.93 | 1 | 31220 | chr12: | chr12: | OAS3 | snpAtOE |
| | | | | | | 113374038: | 113344413: | | |
| | | | | | | 113376755 | 113346052 | | |
| rs10774671 | 4frag | T | 7.93 | 1 | 31310 | chr12: | chr12: | OAS3 | snpAtOE |
| | | | | | | 113374038: | 113344413: | | |
| | | | | | | 113376755 | 113346052 | | |
| rs10774671 | 4frag | T | 10.57 | 1 | 39560 | chr12: | chr12: | OAS3 | snpAtOE |
| | | | | | | 113374038: | 113414321: | | |
| | | | | | | 113376755 | 113417462 | | |
| rs10774671 | 4frag | T | 10.57 | 1 | 39504 | chr12: | chr12: | OAS3 | snpAtOE |
| | | | | | | 113374038: | 113414321: | | |
| | | | | | | 113376755 | 113417462 | | |
| rs10774671 | 4frag | T | 10.57 | 1 | 39908 | chr12: | chr12: | OAS3 | snpAtOE |
| | | | | | | 113374038: | 113414321: | | |
| | | | | | | 113376755 | 113417462 | | |
| rs10774671 | 4frag | T | 10.57 | 1 | 39572 | chr12: | chr12: | OAS3 | snpAtOE |
| | | | | | | 113374038: | 113414321: | | |
| | | | | | | 113376755 | 113417462 | | |
| rs10774671 | 4frag | T | 10.57 | 1 | 39440 | chr12: | chr12: | OAS3 | snpAtOE |
| | | | | | | 113374038: | 113414321: | | |
| | | | | | | 113376755 | 113417462 | | |
| rs10774671 | 4frag | T | 10.57 | 1 | 39348 | chr12: | chr12: | OAS3 | snpAtOE |
| | | | | | | 113374038: | 113414321: | | |
| | | | | | | 113376755 | 113417462 | | |
| rs10774671 | 4frag \| | T | 8.905 | 1 | 39564 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 8.905 | 1 | 39507 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 8.905 | 1 | 39912 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 8.905 | 1 | 39575 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 8.905 | 1 | 39443 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 11.535 | 1 | 39564 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 11.535 | 1 | 39507 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 11.535 | 1 | 39912 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 11.535 | 1 | 39575 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 11.535 | 1 | 39443 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag \| | T | 11.535 | 1 | 39352 | chr12: | chr12: | OAS3 | snpAtOE |
| | 1frag | | | | | 113374038: | 113414321: | | |
| | | | | | | 113376755 \| | 113417462 \| | | |
| | | | | | | chr12: | chr12: | | |
| | | | | | | 113375558: | 113415286: | | |
| | | | | | | 113376755 | 113416511 | | |
| rs10774671 | 4frag | T | 13.22 | 1 | 39560 | chr12: | chr12: | OAS3 | snpAtOE |
| | | | | | | 113374038: | 113414321: | | |
| | | | | | | 113376755 | 113417462 | | |
| rs10774671 | 4frag | T | 13.22 | 1 | 39504 | chr12: | chr12: | OAS3 | snpAtOE |
| | | | | | | 113374038: | 113414321: | | |
| | | | | | | 113376755 | 113417462 | | |
| rs10774671 | 4frag | T | 13.22 | 1 | 39908 | chr12: | chr12: | OAS3 | snpAtOE |
| | | | | | | 113374038: | 113414321: | | |
| | | | | | | 113376755 | 113417462 | | |
| rs10774671 | 4frag | T | 13.22 | 1 | 39572 | chr12: | chr12: | OAS3 | snpAtOE |
| | | | | | | 113374038: | 113414321: | | |
| | | | | | | 113376755 | 113417462 | | |
| rs10774671 | 4frag | T | 13.22 | 1 | 39440 | chr12: | chr12: | OAS3 | snpAtOE |
| | | | | | | 113374038: | 113414321: | | |
| | | | | | | 113376755 | 113417462 | | |
| rs10774671 | 4frag | T | 13.22 | 1 | 39348 | chr12: | chr12: | OAS3 | snpAtOE |
| | | | | | | 113374038: | 113414321: | | |
| | | | | | | 113376755 | 113417462 | | |
| rs10774671 | 4frag | T | 10.65 | 1 | 39560 | chr12: | chr12: | OAS3 | snpAtOE |
| | | | | | | 113374038: | 113414321: | | |
| | | | | | | 113376755 | 113417462 | | |
| rs10774671 | 4frag | T | 10.65 | 1 | 39504 | chr12: | chr12: | OAS3 | snpAtOE |
| | | | | | | 113374038: | 113414321: | | |
| | | | | | | 113376755 | 113417462 | | |
| rs10774671 | 4frag | T | 10.65 | 1 | 39908 | chr12: | chr12: | OAS3 | snpAtOE |
| | | | | | | 113374038: | 113414321: | | |
| | | | | | | 113376755 | 113417462 | | |
| rs10774671 | 4frag | T | 10.65 | 1 | 39572 | chr12: | chr12: | OAS3 | snpAtOE |
| | | | | | | 113374038: | 113414321: | | |
| | | | | | | 113376755 | 113417462 | | |
| rs10774671 | 4frag | T | 10.65 | 1 | 39440 | chr12: | chr12: | OAS3 | snpAtOE |
| | | | | | | 113374038: | 113414321: | | |
| | | | | | | 113376755 | 113417462 | | |
| rs10774671 | 4frag | T | 10.65 | 1 | 39348 | chr12: | chr12: | OAS3 | snpAtOE |
| | | | | | | 113374038: | 113414321: | | |
| | | | | | | 113376755 | 113417462 | | |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs10774671 | NA | NA | NA | NA | NA | NA | NA | NA | snpAtPromoter |
| rs2109069 | 4frag \| | T | 6.58 | 1 | 83762 | chr19: | chr19: | DPP9 | snpAtOE |
| | 1frag | | | | | 4723161: | 4638892: | | |
| | | | | | | 4724456 \| | 4641573 \| | | |
| | | | | | | chr19: | chr19: | | |
| | | | | | | 4723293: | 4638982: | | |
| | | | | | | 4724035 | 4640184 | | |
| rs2109069 | 4frag \| | T | 7.87 | 1 | 67918 | chr19: | chr19: | DPP9 | snpAtOE |
| | 1frag | | | | | 4723161: | 4790811: | | |
| | | | | | | 4724456 \| | 4792357 \| | | |
| | | | | | | chr19: | chr19: | | |
| | | | | | | 4723293: | 4791375: | | |
| | | | | | | 4724035 | 4791808 | | |
| rs2109069 | 4frag \| | T | 5.97 | 1 | 67918 | chr19: | chr19: | DPP9 | snpAtOE |
| | 1frag | | | | | 4723161: | 4790811: | | |
| | | | | | | 4724456 \| | 4792357 \| | | |
| | | | | | | chr19: | chr19: | | |
| | | | | | | 4723293: | 4791375: | | |
| | | | | | | 4724035 | 4791808 | | |
| rs2109069 | 4frag \| | T | 13.83 | 1 | 67918 | chr19: | chr19: | DPP9 | snpAtOE |
| | 1frag | | | | | 4723161: | 4790811: | | |
| | | | | | | 4724456 \| | 4792357 \| | | |
| | | | | | | chr19: | chr19: | | |
| | | | | | | 4723293: | 4791375: | | |
| | | | | | | 4724035 | 4791808 | | |
| rs2109069 | 4frag \| | T | 23.7 | 1 | 67918 | chr19: | chr19: | DPP9 | snpAtOE |
| | 1frag | | | | | 4723161: | 4790811: | | |
| | | | | | | 4724456 \| | 4792357 \| | | |
| | | | | | | chr19: | chr19: | | |
| | | | | | | 4723293: | 4791375: | | |
| | | | | | | 4724035 | 4791808 | | |
| rs2109069 | 4frag \| | T | 8.67 | 1 | 67918 | chr19: | chr19: | DPP9 | snpAtOE |
| | 1frag | | | | | 4723161: | 4790811: | | |
| | | | | | | 4724456 \| | 4792357 \| | | |
| | | | | | | chr19: | chr19: | | |
| | | | | | | 4723293: | 4791375: | | |
| | | | | | | 4724035 | 4791808 | | |
| rs2109069 | NA | NA | NA | NA | NA | NA | NA | NA | snpAtPromoter |
| rs2109069 | NA | NA | NA | NA | NA | NA | NA | NA | snpAtPromoter |
| rs2109069 | NA | NA | NA | NA | NA | NA | NA | NA | snpAtPromoter |
| rs2109069 | NA | NA | NA | NA | NA | NA | NA | NA | snpAtPromoter |
| rs2109069 | NA | NA | NA | NA | NA | NA | NA | NA | snpAtPromoter |
| rs2109069 | NA | NA | NA | NA | NA | NA | NA | NA | snpAtPromoter |
| rs77534576 | 4frag | F | 9.93 | 1 | 123944 | chr17: | chr17: | NA | snpAtOE |
| | | | | | | 47949897: | 48072344: | | |
| | | | | | | 47950868 | 48076203 | | |

*biotype for each sentinel is protein coding
**T is true; F is false

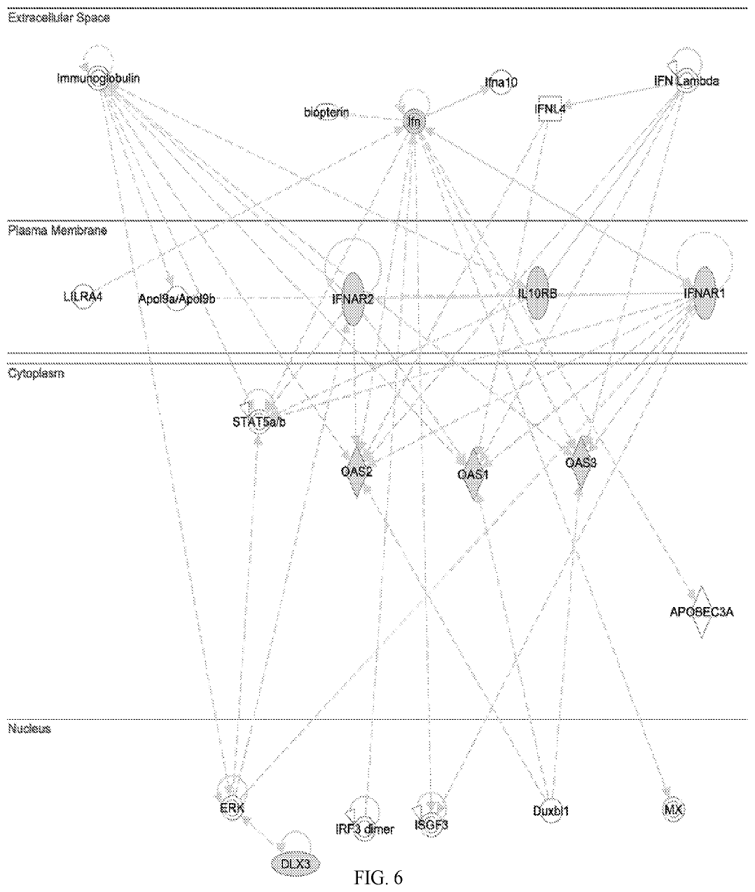

Gene ontology analyses of the set of COVID-19 variant-connected genes showed enrichment for pathways involved in coronavirus pathogenesis (−log P=8.97), viral hypercytokinemia (−log P=8.95), viral/bacterial pattern recognition (−log P=4.0), interferon signaling (−log P=5.93), and T cell exhaustion (−log P=2.39). This set of genes was also enriched (P<0.0003) for factors involved in viral infection, RNA virus replication, anti-viral response, multiple sclerosis, psoriasis, and necrosis (FIG. 6). We note that our approach did not implicate genes for two COVID-19-associated GWAS signals (rs10490770 & rs74956615). rs10490770 is the highest signal located on chromosome 3, and its proxy SNP rs17713054 has been reported to contact the LZTFL1 promoter in lung epithelial cells, suggesting that these risk loci act outside the immune cells³².

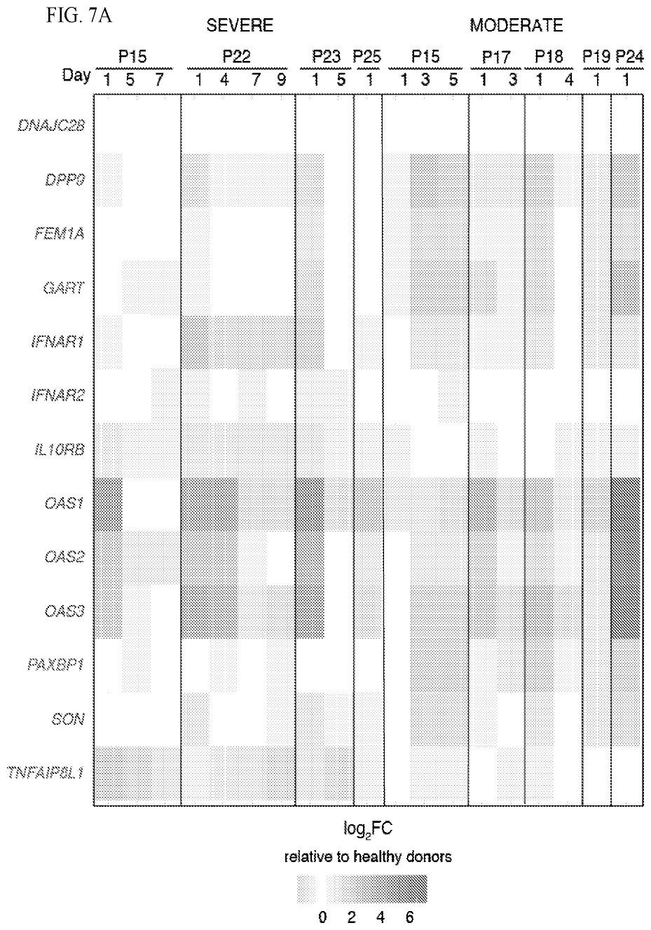

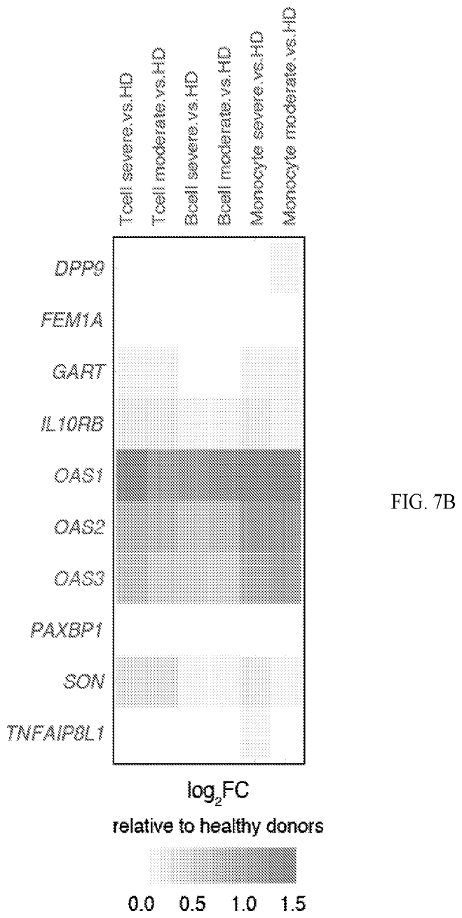

To explore the functional significance of genes implicated through their connection to COVID-19-associated immune regulatory architectures, we compared their expression in bulk blood leukocytes³³ or at the single-cell level³⁴ from COVID-19 patients vs. healthy donors from published datasets. We found nearly two-thirds (10/16) of these implicated genes were differentially expressed in circulating leukocytes of SARS-CoV-2-infected humans (FIGS. 7A and 7B). OAS1, OAS2, OAS3, IL10RB, FEM1A, GART, SON, and IFNAR2 were significantly (FDR<0.05) upregulated in lymphocytes and monocytes from COVID-19 patients compared to healthy controls, while TNFAIP8L1 was significantly downregulated in monocytes from COVID-19 patients (FIGS. 7A and 7B). Importantly, six of these genes also exhibited severity-associated patterns of expression. IFNAR2 was significantly upregulated in severe COVID-19 patients (FDR=0.0075, FIG. 7A), and both OAS1 and OAS3 were upregulated in T cells from severe COVID-19 patients compared to those with mild disease (FIG. 7B). PAXBP1 was significantly downregulated (FDR=0.0067) in severe vs. mild COVID patients, and GART and TNFAIP8L1 were nominally downregulated in severe patients (p=0.047, FDR=0.127).

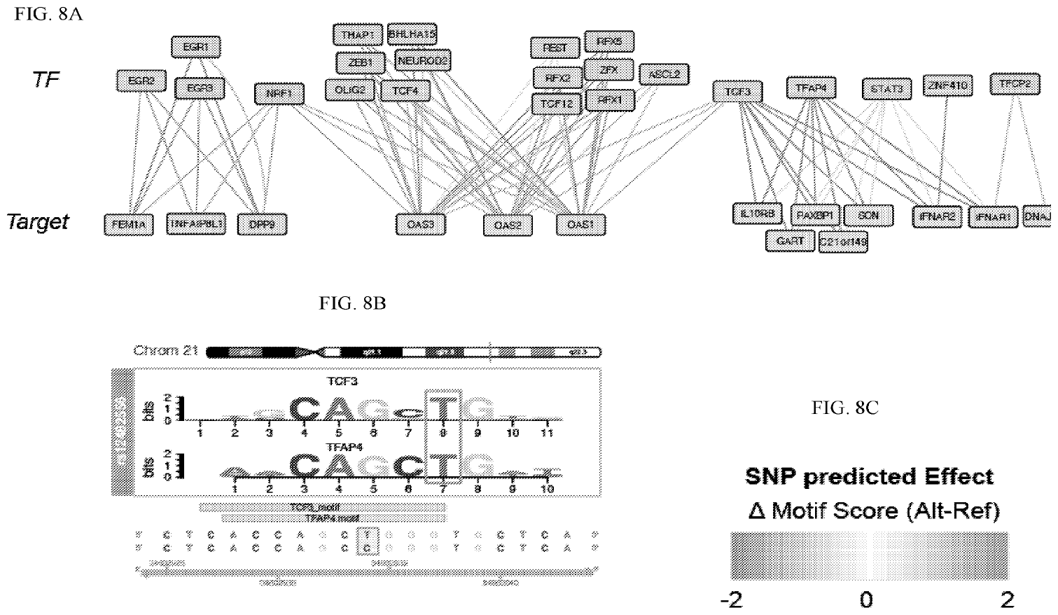

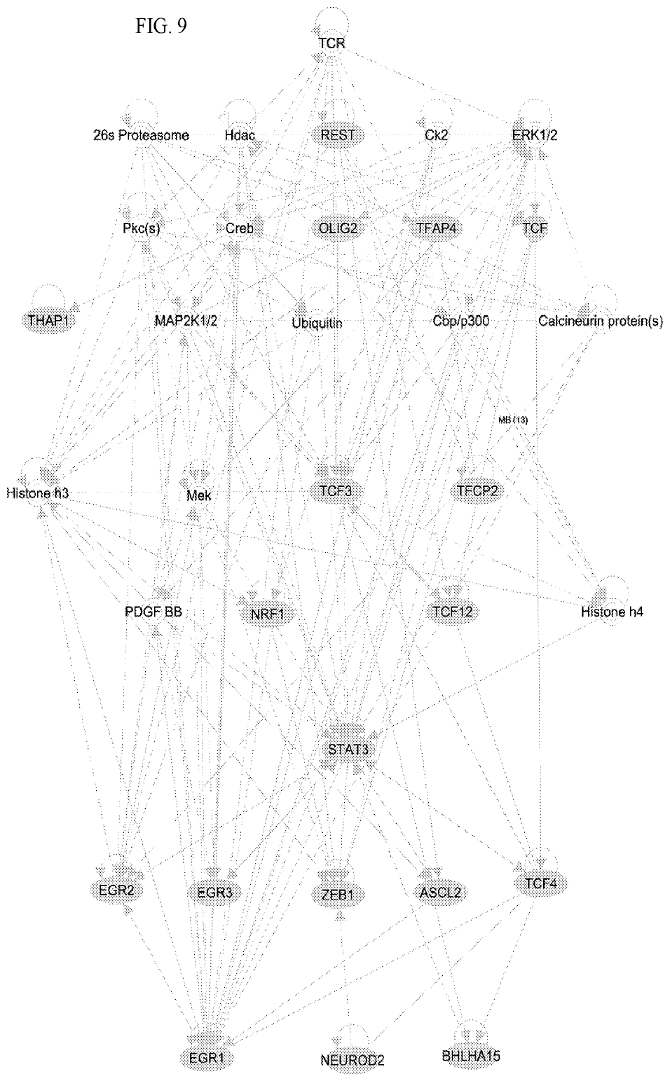

The causal variants identified in this study likely influence COVID-19 risk by directly contacting and altering the expression of their target genes in immune cell types. To test whether these COVID-19 variants may influence gene transcription by affecting the binding of transcription factors, we used a modeling approach to predict their impact on the affinity of transcription factors for consensus DNA motifs present in the regulatory landscape of COVID-19-relevant immune cell types. This approach identified 9 COVID-19-associated SNPs predicted to impact binding of 22 expressed transcription factors to 14 of the 16 genes identified in this study (FIG. 8). COVID-19 risk-associated sequence variation at IFNAR2 may increase binding of ZNF410, a zinc finger protein involved in repression of fetal hemoglobin in erythroid cells³⁵, and risk variants also increase the predicted affinity of STAT3 for elements connected to IFNAR1, IFNAR2, PAXBP1, GART, C21ORF49, SON, and IL10RB. Variants connected to OAS1, OAS2 and OAS3 may affect the binding of 15 distinct transcription factors, including the RFX family of transcription factors involved in expression of MHC class II genes, and the plasmablast and TFH factors MIST1 (BHLHA15)³⁶ and ASCL2³⁷. This set of affected transcription factors form a network of highly co-regulated activities downstream of the TCR, particularly centered around STAT3 and EGR1 (FIG. 9), and enriched for roles in hematopoietic, B and T cell development, differentiation, and function.

Together, our results suggest that genes central to viral genome sensing, host control of viral replication, the interferon response, and immune inflammation are likely under genetic control by common variants associated with COVID-19 disease risk. This study implicated multiple genes at all but one locus. While methods for fine-mapping GWAS signals generally assume a single causal variant acting at one effector gene, this assumption is not always valid. We observe clear evidence for pleiotropic effects of COVID-19 disease risk-associated genetic variation at the level of multiple proxies in open chromatin at each locus in the same and/or distinct cell types, and individual accessible proxies contacting multiple genes in one or across more cell types (FIG. 5). Similar pleiotropy was observed for FTO obesity variants on the dynamics and lineage-specific expression of distal genes such as IRX3 and IRX5³⁸.

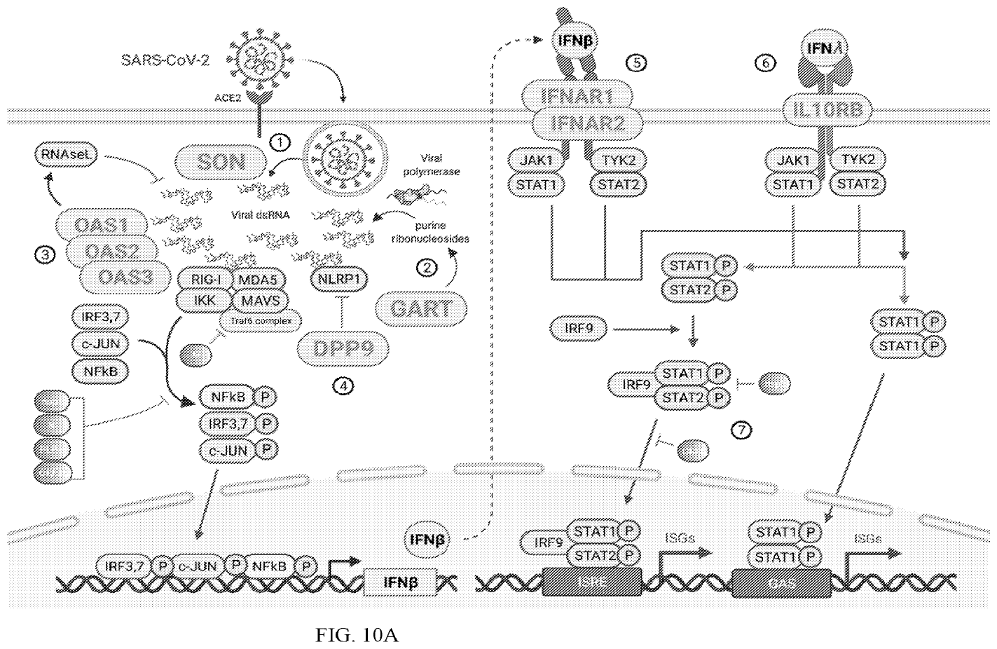

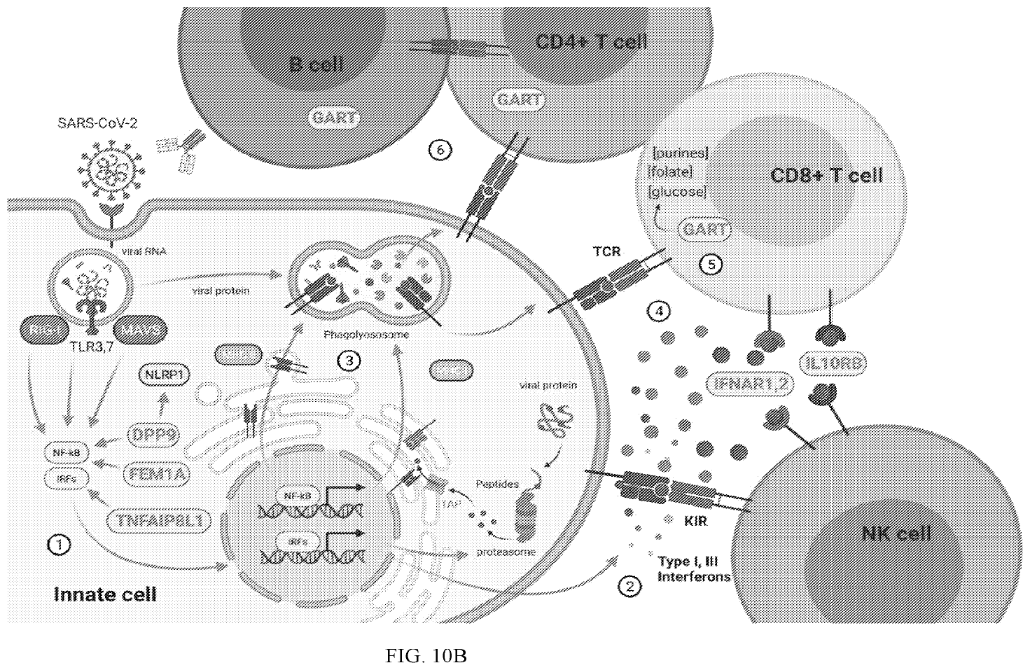

The genes identified through physical association with accessible COVID-19 variants have known roles in viral replication, the interferon response, and inflammation. The genes GART and SON encode factors that may directly impact SARS-CoV-2 replication (FIG. 10A). SON encodes a factor that regulates HBV influenza A replication^{39,40}, while GART controls de novo purine pools required for coronavirus RNA replication that may also drive evolution of viral variants over the course of the pandemic^{41,42}. Interferons (IFN) are important for the control of early virus replication and in determining moderate vs. severe inflammatory disease⁴³. SARS-CoV2 induces type I and type III interferons⁴⁴ that signal through IFNAR1, IFNAR2, and IL10RB (FIG. 10A), but SARS-CoV2 also encodes factors that can inhibit type I and III responses⁴⁵⁻⁴⁷. Thus, many SARS-CoV-2-infected individuals exhibit blunted and/or delayed interferon responses^{20,48-50}, and experience more severe disease than COVID-19 patients with strong interferon responses^{32,50,51} SARS-CoV-2 dsRNA genomes are sensed by the RIG-I/MDA5 and RNAseL pathways^{52,53}. OAS1, OAS2, and OAS3 encode crucial regulators of dsRNA degradation by RNAseL, and DPP9 regulates the activity of NLRP1, a dsRNA-sensing component of the inflammasome⁵⁴ (FIG. 10A). Gain of function mutations in OAS1 lead to autoinflammatory disease in humans⁵⁵, polymorphisms at the OAS1 locus are associated with type 2 diabetes⁵⁶, a pre-existing condition associated with severe COVID-19 disease⁵⁷, and genetic variation at DPP9 is associated with the risk of developing pulmonary fibrosis⁵⁸. Cytokine release syndrome is a major inflammatory complication in patients with severe COVID-19 disease⁵⁹⁻⁶¹. Receptors for type I (IFNAR1 and 2) and III (IL10RB) interferons drive inflammation mediated by NK and CD8+ T cells, and IL-10RB binds IL-10 whose levels are a severity predictor in COVID-19⁶² (FIG. 10B). FEM1A encodes a negative regulator of NFkB activation⁶³, and TNFAIP8L1 regulates expression of the chemokine MCP-164. DNAJC28 is a mitochondrial Hsp40 family member and cofactor of Hsp70 heat shock proteins⁶⁵. PAXBP1 encodes a regulator of ROS and p53⁶⁶, and DLX3 encodes a homeobox protein known to function downstream of the TGFB, BMP, and WNT pathways in tooth and placental development⁶⁷, but immune roles for these factors have not been established.

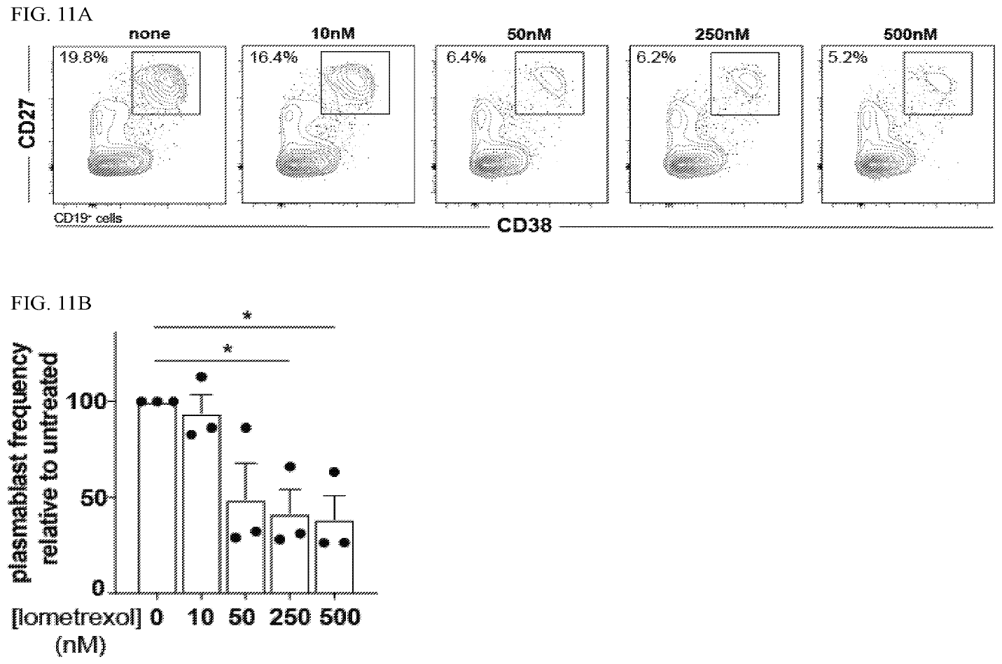

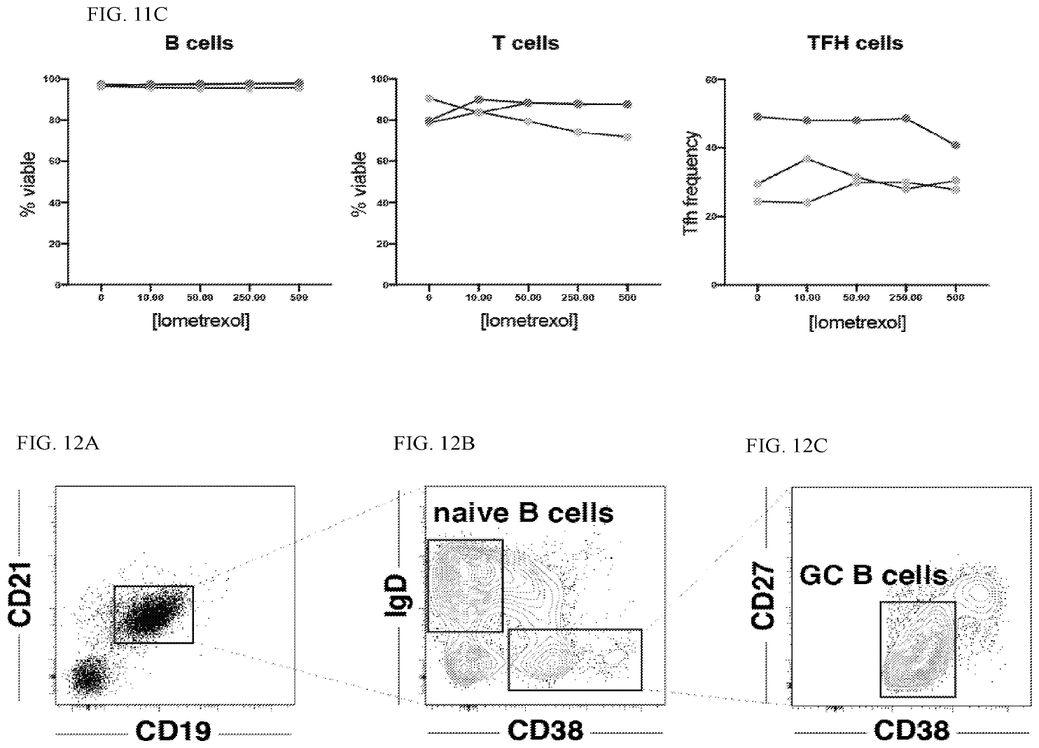

GART encodes an enzyme involved in purine biosynthesis, and its folate-derived metabolites have roles in DNA methylation and mitochondrial redox, processes that regulate immune cell function⁶⁸ (FIG. 10B). To test for a role for GART in adaptive immune responses associated with susceptibility to severe COVID-19, we used the GART inhibitory drug lometrexol in an in vitro human tonsillar organoid model of T cell-dependent germinal center B cell differentiation⁶⁹. After 7 days in culture, T-B interactions in control organoids supported the differentiation of CD27+CD38+ GCB cell plasmablasts (FIGS. 11A and 11B) capable of producing high-affinity class-switched antibodies in this model⁶⁹. The GART inhibitor lometrexol abrogated plasmablast differentiation in a dose-dependent manner (FIGS. 11A and 11B) without affecting B or T cell survival or TFH frequency (FIG. 11C). These results indicate that GART has a previously unappreciated role in T cell-B cell germinal center reactions, and further link GART to immune processes associated with COVID-19 disease severity.

Immune responses to infection can lead to protection from disease or pathology due to excessive inflammation, and autoimmune disorders like systemic lupus crythematosus (SLE), rheumatoid arthritis (RA) and ulcerative colitis (UC) are the result of inappropriate immune responses against self or harmless antigens. This balance is determined by the differentiation and function of T cells and their orchestrated collaboration with other cells of the immune system.

Genome-wide association studies (GWAS) have demonstrated that the balance between appropriate and pathologic immune responses in autoimmune disease and the infectious disease COVID-19 are under genetic control. To provide mechanistic insight into the function of disease-associated genetic variation, we describe 3D/spatial genomic maps of COVID-19 variant accessibility and gene connectivity in human follicular T cells herein, and we have also generated new maps of autoimmune disease variant and gene connectivity in activated CD4+ T cells (See the world wide web at biorxiv.org/content/10.1101/2023.04.05.535731v1). These ‘variant-to-gene’ maps can implicate factors with no prior known role in immunity or autoimmune disease, many of which are druggable.

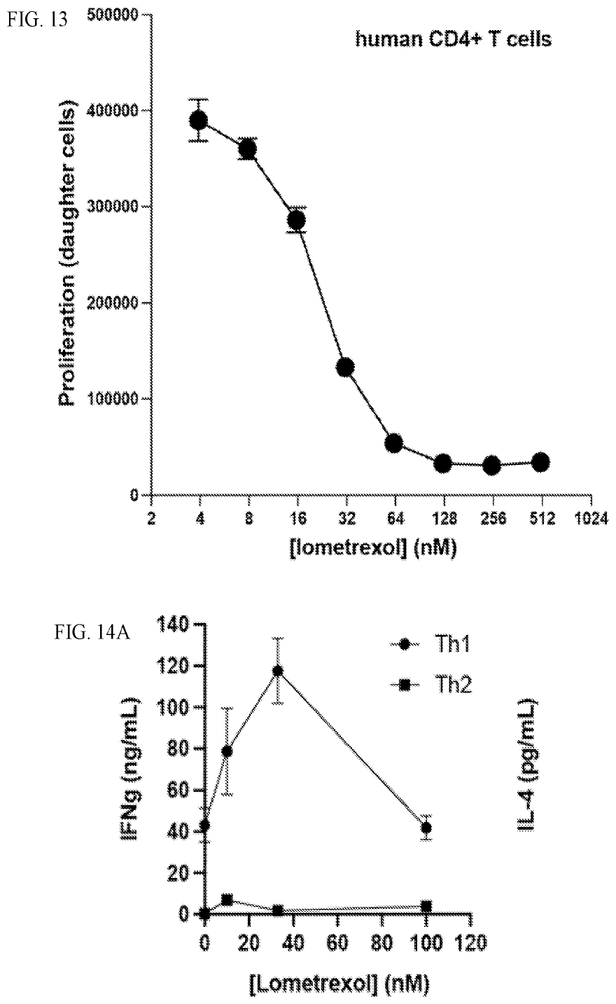

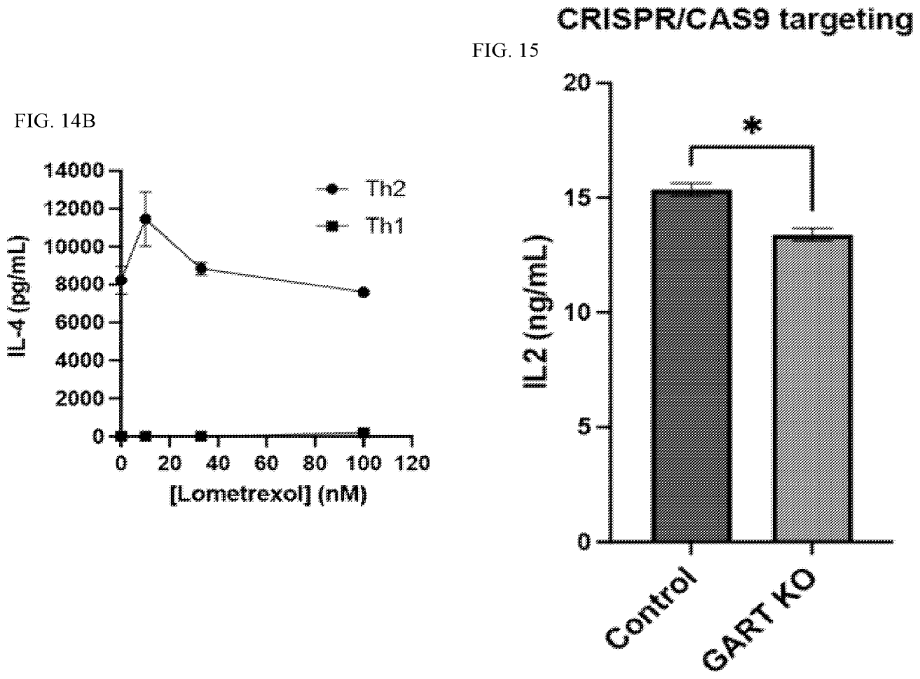

As described herein, GART, a novel target has been identified through association with non-coding disease SNPs. GART is an enzyme in the de novo purine nucleoside pathway. This gene was implicated in the new maps through physical contact with a variant associated with UC disease risk (See the world wide web at biorxiv.org/content/10.1101/2023.04.05.535731v1). We discovered that the GART inhibitory drug lometrexol blocks T cell-B cell collaboration in a tonsil organoid system of humoral immune responses. The data presented in FIG. 13 demonstrates that lometrexol can inhibit the proliferation of activated human CD4+ T cells in vitro (FIG. 13), but at low doses enhances the production of IFNγ and IL-4 from Th1 and Th2 cells, respectively (FIG. 14).

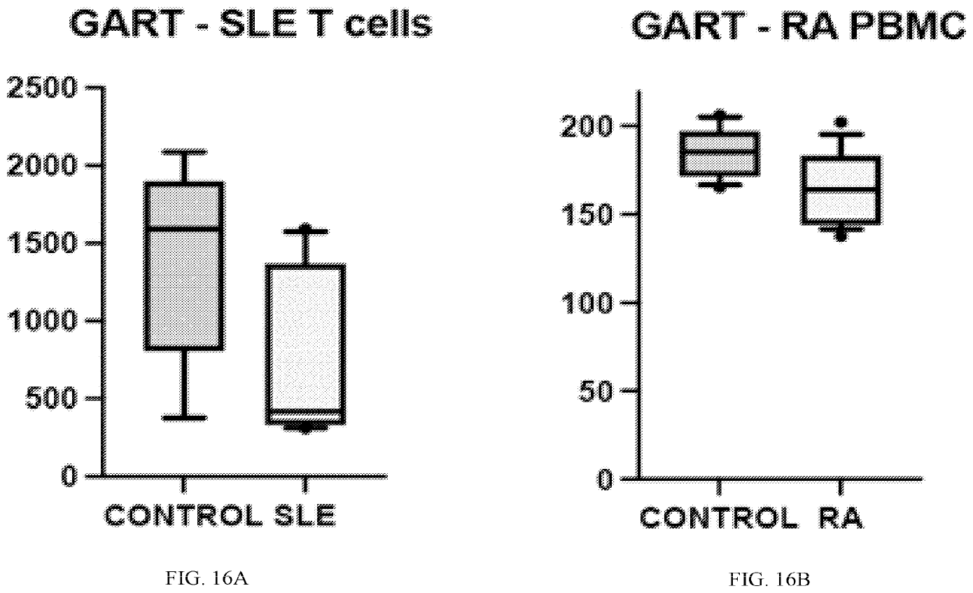

In a separate approach to induce loss of GART function, we delivered gRNA-Cas9 ribonucleoprotein complexes targeting the GART gene to human CD4+ T cells by nucleofection. Upon stimulation through the TCR and CD28, GART-targeted T cells exhibited reduced secretion of IL-2 compared to control cells transfected with Cas9 alone (FIG. 15). Mining of public gene expression data shows that GART expression is significantly decreased in T cells from the peripheral blood of patients with severe COVID-19 (see FIG. 3A in M. Pahl et al. 2022), SLE (FIG. 16A), and RA (FIG. 16B), further strengthening the link between GART and T cell-mediated immunopathology.

In another approach, we assessed the effects of the GART inhibitory drugs Pemetrexed and Perlitrexol. See the world wide web at //go.drugbank.com/drugs/DB00642 and //go.drugbank.com/drugs/DB 12757, respectively. Pemetrexed is an antifolate containing the pyrrolopyrimidine-based nucleus that exerts its antineoplastic activity by disrupting folate-dependent metabolic processes essential for cell replication. In vitro studies have shown that pemetrexed inhibits thymidylate synthase (TS), dihydrofolate reductase (DHFR), and glycinamide ribonucleotide formyltransferase (GARFT), all folate-dependent enzymes involved in the de novo biosynthesis of thymidine and purine nucleotides. Pemetrexed is transported into cells by both the reduced folate carrier and membrane folate binding protein transport systems. Once in the cell, pemetrexed is converted to polyglutamate forms by the enzyme folylpolyglutamate synthetase. The polyglutamate forms are retained in cells and are inhibitors of TS and GARFT. Polyglutamation is a time- and concentration-dependent process that occurs in tumor cells and, to a lesser extent, in normal tissues. Polyglutamated metabolites have an increased intracellular half-life resulting in prolonged drug action in malignant cells.”

Pemetrexed has been safely used in the clinic. These drugs should be efficacious for reducing T cell-mediated inflammation and enhance B cell antibody production, which would be beneficial in autoimmune disorders mediated by auto-antibodies, and also in infectious or autoinflammatory diseases where the goal is to limit immune-mediated inflammation.

This work implicates genetic variation in the cis-regulatory architecture of immune cells as a contributor to the observed variation in COVID-19 disease severity. The initial GWAS implicated candidate effector genes using metrics of linear proximity to the GWAS signal and public expression quantitative trait loci (eQTLs) datasets²⁵. Several of our V2G attributions agreed with these analyses, including OAS1, OAS2, and OAS3 as the likely effector genes for rs10774671. DLX3, IL10RB, and IFNAR2 were also implicated in the prior study via intersection with eQTLs from various tissues and cell types. However, a number of GWAS candidate genes were not implicated in our study, and our immune-focused PCC maps identified several candidate effectors not implicated previously: IFNAR1, GART, SON, and AP00295.9 for rs1305728 and TNFAIP8L1 and FEM1A for rs77534576. In addition, our V2G mapping of genes involved in COVID severity identified GART as a novel target whose activity could be increased for better anti-viral humoral immune responses or inhibited as a potential treatment for systemic autoimmune disease.

Importantly, T cell-B cell collaboration for antibody production is also involved in the generation of disease-causing auto-antibodies in autoimmune disorders such as Lupus, Myasthenia Gravis, Type 1 diabetes, etc. In this case therapeutics that inhibit GART function could reduce pathogen autoantibody levels and ameliorate disease.

### Example II

### Methods for Diagnosis of Inflammatory Disorders and Screening Assays to Identify Therapeutic Agents Useful for the Treatment of the Same

The information herein above can be applied clinically to patients for diagnosing the presence of, or an increased susceptibility for developing severe Covid and other inflammatory disorders, and for therapeutic intervention. A preferred embodiment of the invention comprises clinical application of the information described herein to a patient. Diagnostic compositions, including microarrays. and methods can be designed to identify the gene targets and appropriate therapeutic as described herein in nucleic acids from a patient to assess susceptibility for developing inflammatory disorders, including severe Covid 19. This can occur after a patient arrives in the clinic; the patient has blood drawn, and using the diagnostic methods described herein, a clinician can detect the SNPs in the chromosomal regions described herein. The information obtained from the patient sample. which can optionally be amplified prior to assessment, will be used to diagnose a patient with an increased or decreased susceptibility for developing an inflammatory disorder, particularly severe Covid 19. Kits for performing the diagnostic method of the invention are also provided herein. Such kits comprise a microarray comprising at least one of the SNPs provided herein in and the necessary reagents for assessing the patient samples as described above.

Capture C genes associated with severe Covid 19 are described herein. Agents targeting these genes and gene products are also provided in Table 4. In certain embodiments, inhibitors that target each of the upregulated genes are employed. In other embodiments, agents which enhance or augment the function of down modulated genes are employed. For example, phosphoribosylglycinamide formyltransferase is an enzyme which catalyzes a nucleophilic acyl substitution of the formyl group from 10-formyltetrahydrofolate (fTHF) to N1-(5-phospho-D-ribosyl)glycinamide (GAR) to form N2-formyl-N1-(5-phospho-D-ribosyl)glycinamide (fGAR). It uses fTHF and GAR as substrates to generate the products THF (folate) and fGAR—which appear to play an important role in the immune function described herein (i.e., promoting the generation of antibody-producing plasmablasts), thus fTHF and GAR would act as GART agonists. Alternatively, GART can be bypassed by providing its products THF and fGAR, thereby simulating GART activity and act to agonize the downstream processes.

TABLE 4
Genes involved in Inflammatory disorders such as severe Covid 19 and drugs which modulate the same

| gene_ | gene_claim_ | Entrez_ | Interaction_ | drug_claim_ | drug_claim_ | drug_ | drug_ |
|---|---|---|---|---|---|---|---|
| name | name | id | types | name | primary_name | name | chembl_id |
| DPP9 | 2357 | 91039 | inhibitor | 8551 | 1G244 | | |
| DPP9 | 2357 | 91039 | inhibitor | 8550 | COMPOUND | DESERPIDINE | CHEMBL1200515 |
| | | | | | 24DD [PMID: | | |
| | | | | | 20684603] | | |
| GART | PGFT | 2618 | inhibitor | CHEMBL1200373 | PEMETREXED | PEMETREXED | CHEMBL1200373 |
| | | | | | DISODIUM | DISODIUM | |
| GART | P22102 | 2618 | | PEMETREXED | PEMETREXED | PEMETREXED | CHEMBL1201258 |
| | | | | | | (CHEMBL1201258) | |
| GART | P22102 | 2618 | | PEMETREXED | PEMETREXED | PEMETREXED | CHEMBL1201258 |
| | | | | INTERFERON | INTERFERON | (CHEMBL1201258) | |
| IFNAR1 | P48551 | 3454 | | ALFA-2A, | ALFA-2A, | INTERFERON | CHEMBL2108508 |
| | | | | RECOMBINANT | RECOMBINANT | ALFA-2A | |
| IFNAR1 | P48551 | 3454 | | PEG- | PEG- | | |
| | | | | INTERFERON | INTERFERON | | |
| | | | | ALFACON-1 | ALFACON-1 | | |
| IFNAR1 | IFNAR | 3454 | agonist | CHEMBL2109047 | INTERFERON | INTERFERON | CHEMBL2109047 |
| | | | | | ALFA-N3 | ALFA-N3 | |
| IFNAR1 | P17181 | 3454 | | INTERFERON | INTERFERON | INTERFERON | CHEMBL2108509 |
| | | | | ALFA-N1 | ALFA-N1 | ALFA-N1 | |
| IFNAR1 | P17181 | 3454 | | INTERFERON | INTERFERON | INTERFERON | CHEMBL1201563 |
| | | | | BETA-1B | BETA-1B | BETA-1B | |
| IFNAR1 | P48551 | 3454 | | MODIFIED IFN- | MODIFIED IFN- | | |
| | | | | ALPHA | ALPHA | | |
| IFNAR1 | P17181 | 3454 | | RECOMBINANT | RECOMBINANT | | |
| | | | | INTERFERON | INTERFERON | | |
| | | | | ALFA-2B | ALFA-2B | | |
| IFNAR1 | IFNAR | 3454 | agonist | CHEMBL1201563 | INTERFERON | INTERFERON | CHEMBL1201563 |
| | | | | | BETA-1B | BETA-1B | |
| IFNAR1 | P17181 | 3454 | | MODIFIED IFN- | MODIFIED IFN- | | |
| | | | | ALPHA | ALPHA | | |
| IFNAR1 | P48551 | 3454 | | INTERFERON | INTERFERON | INTERFERON | CHEMBL2108510 |
| | | | | BETA | BETA | BETA | |
| IFNAR1 | P17181 | 3454 | | INTERFERON | INTERFERON | INTERFERON | CHEMBL2108510 |
| | | | | BETA | BETA | BETA | |
| IFNAR1 | P17181 | 3454 | | INTERFERON | INTERFERON | INTERFERON | |
| | | | | BETA-1B | BETA-1B | BETA-1B | CHEMBL1201563 |
| IFNAR1 | P17181 | 3454 | | INTERFERON | INTERFERON | INTERFERON | |
| | | | | BETA-1A | BETA-1A | BETA-1A | CHEMBL1201562 |
| IFNAR1 | P17181 | 3454 | | MODIFIED IFN- | MODIFIED IFN- | | |
| | | | | BETA | BETA | | |
| IFNAR1 | IFNAR | 3454 | agonist | CHEMBL1201557 | INTERFERON | INTERFERON | CHEMBL1201557 |
| | | | | | ALFACON-1 | ALFACON-1 | |
| IFNAR1 | P17181 | 3454 | | INTERFERON | INTERFERON | INTERFERON | CHEMBL2108508 |
| | | | | ALFA-2A, | ALFA-2A, | ALFA-2A | |
| | | | | RECOMBINANT | RECOMBINANT | | |
| IFNAR1 | P48551 | 3454 | | PEGINTERFERON | PEGINTERFERON | PEGINTERFERON | CHEMBL1201560 |
| | | | | ALFA-2A | ALFA-2A | ALFA-2A | |
| IFNAR1 | P48551 | 3454 | | INTERFERON | INTERFERON | INTERFERON | CHEMBL2108506 |
| | | | | ALPHA | ALPHA | ALFA | |
| IFNAR1 | IFNAR | 3454 | agonist | CHEMBL2107842 | ALBINTERFERON | ALBINTERFERON | CHEMBL2107842 |
| | | | | | ALFA-2B | ALFA-2B | |
| IFNAR1 | P48551 | 3454 | | INTERFERON | INTERFERON | INTERFERON | CHEMBL1201558 |
| | | | | ALFA-2B, | ALFA-2B, | ALFA-2B | |
| | | | | RECOMBINANT | RECOMBINANT | | |
| IFNAR1 | P48551 | 3454 | | INTERFERON | INTERFERON | INTERFERON | CHEMBL2108509 |
| | | | | ALFA-N1 | ALFA-N1 | ALFA-N1 | |
| IFNAR1 | P17181 | 3454 | | INTERFERON | INTERFERON | INTERFERON | CHEMBL2108508 |
| | | | | ALFA-2A, | ALFA-2A, | ALFA-2A | |
| | | | | RECOMBINANT | RECOMBINANT | | |
| IFNAR1 | P48551 | 3454 | | INTERFERON | INTERFERON | INTERFERON | CHEMBL1201557 |
| | | | | ALFACON-1 | ALFACON-1 | ALFACON-1 | |
| IFNAR1 | P17181 | 3454 | | INTERFERON | INTERFERON | INTERFERON | CHEMBL2108506 |
| | | | | ALPHA | ALPHA | ALFA | |
| IFNAR1 | IFNAR | 3454 | agonist | CHEMBL1201560 | PEGINTERFERON | PEGINTERFERON | CHEMBL1201560 |
| | | | | | ALFA-2A | ALFA-2A | |
| IFNAR1 | P48551 | 3454 | | RECOMBINANT | RECOMBINANT | | |
| | | | | INTERFERON | INTERFERON | | |
| | | | | ALFA-2B | ALFA-2B | | |
| IFNAR1 | P17181 | 3454 | | SIFALIMUMAB | SIFALIMUMAB | SIFALIMUMAB | CHEMBL1743069 |
| IFNAR1 | P48551 | 3454 | | INTERFERON | INTERFERON | INTERFERON | CHEMBL1201563 |
| | | | | BETA-1B | BETA-1B | BETA-1B | |
| IFNAR1 | P17181 | 3454 | | PEGINTERFERON | PEGINTERFERON | PEGINTERFERON | CHEMBL1201560 |
| | | | | ALFA-2A | ALFA-2A | ALFA-2A | |
| IFNAR1 | IFNAR | 3454 | agonist | CHEMBL1201562 | INTERFERON | INTERFERON | CHEMBL1201562 |
| | | | | | BETA-1A | BETA-1A | |
| IFNAR1 | P17181 | 3454 | | PEG- | PEG- | | |
| | | | | INTERFERON | INTERFERON | | |
| | | | | ALFACON-1 | ALFACON-1 | | |
| IFNAR1 | P17181 | 3454 | | INTERFERON | INTERFERON | INTERFERON | CHEMBL1201557 |
| | | | | ALFACON-1 | ALFACON-1 | ALFACON-1 | |
| IFNAR1 | P17181 | 3454 | | INTERFERON | INTERFERON | INTERFERON | CHEMBL1201558 |
| | | | | ALFA-2B, | ALFA-2B, | ALFA-2B | |
| | | | | RECOMBINANT | RECOMBINANT | | |
| IFNAR1 | P17181 | 3454 | | INTERFERON | INTERFERON | INTERFERON | CHEMBL2108509 |
| | | | | ALFA-N1 | ALFA-N1 | ALFA-N1 | |
| IFNAR1 | P48551 | 3454 | | MODIFIED IFN- | MODIFIED IFN- | | |
| | | | | BETA | BETA | | |
| IFNAR1 | P48551 | 3454 | | INTERFERON | INTERFERON | INTERFERON | CHEMBL1201558 |
| | | | | ALPHA-2B | ALPHA-2B | ALFA-2B | |
| IFNAR1 | P17181 | 3454 | | INTERFERON | INTERFERON | INTERFERON | CHEMBL2109047 |
| | | | | ALFA-N3 | ALFA-N3 | ALFA-N3 | |
| IFNAR1 | IFNAR | 3454 | agonist | CHEMBL1201561 | PEGINTERFERON | PEGINTERFERON | CHEMBL1201561 |
| | | | | | ALFA-2B | ALFA-2B | |
| IFNAR1 | P17181 | 3454 | | INTERFERON | INTERFERON | INTERFERON | CHEMBL2109047 |
| | | | | ALFA-N3 | ALFA-N3 | ALFA-N3 | |
| IFNAR1 | P17181 | 3454 | | INTERFERON | INTERFERON | INTERFERON | CHEMBL1201558 |
| | | | | ALFA-2B, | ALFA-2B, | ALFA-2B | |
| | | | | RECOMBINANT | RECOMBINANT | | |
| IFNAR1 | IFNAR | 3454 | agonist | CHEMBL2108677 | PEGINTERFERON | PEGINTERFERON | CHEMBL2108677 |
| | | | | | BETA-1A | BETA-1A | |
| IFNAR1 | P17181 | 3454 | | INTERFERON | INTERFERON | INTERFERON | CHEMBL1201558 |
| | | | | ALPHA-2B | ALPHA-2B | ALFA-2B | |
| IFNAR1 | IFNAR | 3454 | agonist | CHEMBL2108508 | INTERFERON | INTERFERON | CHEMBL2108508 |
| | | | | | ALFA-2A | ALFA-2A | |
| IFNAR1 | IFNAR | 3454 | agonist | CHEMBL1201558 | INTERFERON | INTERFERON | CHEMBL1201558 |
| | | | | | ALFA-2B | ALFA-2B | |
| IFNAR1 | P48551 | 3454 | | INTERFERON | INTERFERON | INTERFERON | CHEMBL1201562 |
| | | | | BETA-1A | BETA-1A | BETA-1A | |
| IFNAR1 | P48551 | 3454 | | INTERFERON | INTERFERON | INTERFERON | CHEMBL2109047 |
| | | | | ALFA-N3 | ALFA-N3 | ALFA-N3 | |
| IFNAR1 | P17181 | 3454 | | INTERFERON | INTERFERON | INTERFERON | CHEMBL1201557 |
| | | | | ALFACON-1 | ALFACON-1 | ALFACON-1 | |
| IFNAR2 | 1898 | 3455 | agonist | 4965 | IFN-CE ± 7 | | |
| IFNAR2 | 1898 | 3455 | agonist | 4964 | IFN-CE ± 6 | | |
| IFNAR2 | IFNABR | 3455 | agonist | CHEMBL1201560 | PEGINTERFERON | PEGINTERFERON | CHEMBL1201560 |
| | | | | | ALFA-2A | ALFA-2A | |
| IFNAR2 | P48551 | 3455 | | INTERFERON | INTERFERON | INTERFERON | CHEMBL1201563 |
| | | | | BETA-1B | BETA-1B | BETA-1B | |
| IFNAR2 | P48551 | 3455 | | INTERFERON | INTERFERON | INTERFERON | CHEMBL1201557 |
| | | | | ALFACON-1 | ALFACON-1 | ALFACON-1 | |
| IFNAR2 | 1898 | 3455 | agonist | 4956 | IFN-CE ± 10 | | |
| IFNAR2 | TTDS00492 | 3455 | binder | DAP001284 | INTERFERON | INTERFERON | CHEMBL1201557 |
| | | | | | ALFACON-1 | ALFACON-1 | |
| IFNAR2 | IFNABR | 3455 | agonist | CHEMBL1201558 | INTERFERON | INTERFERON | CHEMBL1201558 |
| | | | | | ALFA-2B | ALFA-2B | |
| IFNAR2 | IFNABR | 3455 | agonist | CHEMBL1201557 | INTERFERON | INTERFERON | CHEMBL1201557 |
| | | | | | ALFACON-1 | ALFACON-1 | |
| IFNAR2 | IFNABR | 3455 | agonist | CHEMBL2108677 | PEGINTERFERON | PEGINTERFERON | CHEMBL2108677 |
| | | | | | BETA-1A | BETA-1A | |
| IFNAR2 | IFNABR | 3455 | agonist | CHEMBL1201563 | INTERFERON | INTERFERON | CHEMBL1201563 |
| | | | | | BETA-1B | BETA-1B | |
| IFNAR2 | TTDS00492 | 3455 | binder | DAP001285 | INTERFERON | | |
| | | | | | ALFA-2B, | | |
| | | | | | RECOMBINANT | | |
| IFNAR2 | P48551 | 3455 | | INTERFERON | INTERFERON | INTERFERON | CHEMBL2108508 |
| | | | | ALFA-2A, | ALFA-2A, | ALFA-2A | |
| | | | | RECOMBINANT | RECOMBINANT | | |
| IFNAR2 | TTDS00492 | 3455 | binder | DAP001283 | INTERFERON | INTERFERON | CHEMBL1201563 |
| | | | | | BETA-1B | BETA-1B | |
| IFNAR2 | 1898 | 3455 | agonist | 4966 | IFN-CE ± 8 | | |
| IFNAR2 | 1898 | 3455 | agonist | 4955 | IFN-CE ± 1/13 | | |
| IFNAR2 | TTDS00492 | 3455 | antibody | DCL000623 | RHUMAB IFN | RONTALIZUMAB | CHEMBL1743066 |
| | | | | | ALPHA | | |
| IFNAR2 | P48551 | 3455 | | INTERFERON | INTERFERON | INTERFERON | CHEMBL2108509 |
| | | | | ALFA-N1 | ALFA-N1 | ALFA-N1 | |
| IFNAR2 | 1898 | 3455 | agonist | 4970 | IFN-ceâ | | |
| IFNAR2 | 1898 | 3455 | agonist | 4959 | IFN-CE ± 17 | | |
| IFNAR2 | IFNABR | 3455 | agonist | CHEMBL2107842 | ALBINTERFERON | ALBINTERFERON | CHEMBL2107842 |
| | | | | | ALFA-2B | ALFA-2B | |
| IFNAR2 | 1898 | 3455 | agonist | 4962 | IFN-CE ± 4 | | |
| IFNAR2 | 1898 | 3455 | agonist | 4957 | IFN-CE ± 14 | | |
| IFNAR2 | TTDS00492 | 3455 | antibody | DCL000640 | SIFALIMUMAB | SIFALIMUMAB | CHEMBL1743069 |
| IFNAR2 | TTDS00492 | 3455 | binder | DAP001278 | PEGINTERFERON | PEGINTERFERON | CHEMBL1201560 |
| | | | | | ALFA-2A | ALFA-2A | |
| IFNAR2 | TTDS00492 | 3455 | binder | DAP001282 | INTERFERON | INTERFERON | CHEMBL1201562 |
| | | | | | BETA-1A | BETA-1A | |
| IFNAR2 | 1898 | 3455 | agonist | 4960 | IFN-CE ± 2 | | |
| IFNAR2 | TTDS00492 | 3455 | binder | DAP001279 | PEGINTERFERON | PEGINTERFERON | CHEMBL1201561 |
| | | | | | ALFA-2B | ALFA-2B | |
| IFNAR2 | 1898 | 3455 | agonist | 4961 | IFN-CE ± 21 | | |
| IFNAR2 | IFNABR | 3455 | agonist | CHEMBL1201561 | PEGINTERFERON | PEGINTERFERON | CHEMBL1201561 |
| | | | | | ALFA-2B | ALFA-2B | |
| IFNAR2 | P48551 | 3455 | | INTERFERON | INTERFERON | INTERFERON | CHEMBL1201558 |
| | | | | ALFA-2B, | ALFA-2B, | ALFA-2B | |
| | | | | RECOMBINANT | RECOMBINANT | | |
| IFNAR2 | IFNABR | 3455 | agonist | CHEMBL1201562 | INTERFERON | INTERFERON | CHEMBL1201562 |
| | | | | | BETA-1A | BETA-1A | |
| IFNAR2 | 1898 | 3455 | agonist | 4967 | IFN-CE≤ | | |
| IFNAR2 | 1898 | 3455 | agonist | 4969 | IFN-CE∫ | | |
| IFNAR2 | P48551 | 3455 | | INTERFERON | INTERFERON | INTERFERON | CHEMBL2109047 |
| | | | | ALFA-N3 | ALFA-N3 | ALFA-N3 | |
| IFNAR2 | 1898 | 3455 | agonist | 4963 | IFN-CE ± 5 | | |
| IFNAR2 | TTDS00492 | 3455 | binder | DAP001280 | INTERFERON | INTERFERON | CHEMBL2108509 |
| | | | | | ALFA-N1 | ALFA-N1 | |
| IFNAR2 | IFNABR | 3455 | agonist | CHEMBL2109047 | INTERFERON | INTERFERON | CHEMBL2109047 |
| | | | | | ALFA-N3 | ALFA-N3 | |
| IFNAR2 | 1898 | 3455 | agonist | 4958 | IFN-CE ± 16 | | |
| IFNAR2 | 1898 | 3455 | agonist | 7462 | PEGINTERFERON | PEGINTERFERON | CHEMBL1201561 |
| | | | | | ALFA-2B | ALFA-2B | |
| IFNAR2 | TTDS00492 | 3455 | binder | DAP001281 | INTERFERON | INTERFERON | CHEMBL2109047 |
| | | | | | ALFA-N3 | ALFA-N3 | |
| IFNAR2 | IFNABR | 3455 | agonist | CHEMBL2108508 | INTERFERON | INTERFERON | CHEMBL2108508 |
| | | | | | ALFA-2A | ALFA-2A | |
| IFNAR2 | TTDS00492 | 3455 | stimulator | DCL000688 | ALFA- | INTERFERON | CHEMBL2108509 |
| | | | | | INTERFERON | ALFA-N1 | |
| IFNAR2 | TTDS00492 | 3455 | antagonist | DCL000432 | ANTI-IFN ALFA | | |
| IL10RB | CRFB4 | 3588 | agonist | CHEMBL2108723 | PEGINTERFERON | PEGINTERFERON | CHEMBL2108723 |
| | | | | | LAMBDA-1A | LAMBDA-1A | |
| IL10RB | Q08334 | 3588 | | PEG- | PEG- | | |
| | | | | INTERFERON | INTERFERON | | |
| | | | | LAMBA | LAMBA | | |

### REFERENCES

1. Braun, J. et al. SARS-CoV-2-reactive T cells in healthy donors and patients with COVID-19. Nature 1-8 (2020) doi:10.1038/s41586-020-2598-9.
2. Grifoni, A. et al. Targets of T Cell Responses to SARS-CoV-2 Coronavirus in Humans with COVID-19 Disease and Unexposed Individuals. Cell 181, 1489-1501.e15 (2020).
3. Tan, L. et al. Lymphopenia predicts disease severity of COVID-19: a descriptive and predictive study. Signal Transduct Target Ther 5, 33 (2020).
4. Chen, Z. & Wherry, E. J. T cell responses in patients with COVID-19. Nat Rev Immunol 20, 529-536 (2020).
5. Huang, C. et al. Clinical features of patients infected with 2019 novel coronavirus in Wuhan, China. Lancet 395, 497-506 (2020).
6. Marcos-Jiménez, A. et al. Deregulated cellular circuits driving immunoglobulins and complement consumption associate with the severity of COVID-19 patients. Eur J Immunol (2021) doi:10.1002/eji.202048858.
7. Yang, Y. et al. Exuberant elevation of IP-10, MCP-3 and IL-lra during SARS-CoV-2 infection is associated with disease severity and fatal outcome. (n.d.) doi:10.1101/2020.03.02.20029975.
8. Li, X. et al. Risk factors for severity and mortality in adult COVID-19 inpatients in Wuhan. J Allergy Clin Immun 146, 110-118 (2020).
9. Chua, R. L. et al. COVID-19 severity correlates with airway epithelium-immune cell interactions identified by single-cell analysis. Nat Biotechnol 38, 970-979 (2020).
10. Liao, M. et al. Single-cell landscape of bronchoalveolar immune cells in patients with COVID-19. Nat Med 26, 842-844 (2020).
11. Moderbacher, C. R. et al. Antigen-Specific Adaptive Immunity to SARS-CoV-2 in Acute COVID-19 and Associations with Age and Disease Severity. Cell 183, 996-1012.e19 (2020).
12. Sekine, T. et al. Robust T Cell Immunity in Convalescent Individuals with Asymptomatic or Mild COVID-19. Cell 183, 158-168.e14 (2020).
13. Catanzaro, M. et al. Immune response in COVID-19: addressing a pharmacological challenge by targeting pathways triggered by SARS-CoV-2. Signal Transduct Target Ther 5, 84 (2020).
14. Grant, R. A. et al. Circuits between infected macrophages and T cells in SARS-CoV-2 pneumonia. Nature 1-10 (2021) doi:10.1038/s41586-020-03148-w.
15. Crotty, S. T Follicular Helper Cell Biology: A Decade of Discovery and Diseases. Immunity 50, 1132-1148 (2019).
16. Gaebler, C. et al. Evolution of antibody immunity to SARS-CoV-2. Nature 1-10 (2021) doi:10.1038/s41586-021-03207-w.
17. Mathew, D. et al. Deep immune profiling of COVID-19 patients reveals distinct immunotypes with therapeutic implications. Science 369, eabc8511 (2020).
18. Juno, J. A. et al. Humoral and circulating follicular helper T cell responses in recovered patients with COVID-19. Nat Med 26, 1428-1434 (2020).
19. Yang, X. et al. Naturally activated adaptive immunity in COVID-19 patients. J Cell Mol Med 24, 12457-12463 (2020).
20. Arunachalam, P. S. et al. Systems biological assessment of immunity to mild versus severe COVID-19 infection in humans. Science 369, 1210-1220 (2020).
21. Lakshmanappa, Y. S. et al. SARS-CoV-2 induces robust germinal center CD4 T follicular helper cell responses in rhesus macaques. Nat Commun 12, 541 (2021).
22. Zhang, J. et al. Spike-specific circulating T follicular helper cell and cross-neutralizing antibody responses in COVID-19-convalescent individuals. Nat Microbiol 6, 51-58 (2021).
23. Kuri-Cervantes, L. et al. Comprehensive mapping of immune perturbations associated with severe COVID-19. Sci Immunol 5, eabd7114 (2020).
24. Pairo-Castineira, E. et al. Genetic mechanisms of critical illness in Covid-19. Nature (2020) doi:10.1038/s41586-020-03065-y.
25. COVID-19 Host Genetics Initiative. Mapping the human genetic architecture of COVID-19. Nature (2021) doi: 10.1038/s41586-021-03767-x.
26. Su, C. et al. Mapping effector genes at lupus GWAS loci using promoter Capture-C in follicular helper T cells. Nat Commun 11, 3294 (2020).
27. Pahl, M. C. et al. Cis-regulatory architecture of human ESC-derived hypothalamic neuron differentiation aids in variant-to-gene mapping of relevant complex traits. Biorxiv 2020.07.06.146951 (2020) doi:10.1101/2020.07.06.146951.
28. Chesi, A. et al. Genome-scale Capture C promoter interactions implicate effector genes at GWAS loci for bone mineral density. Nat Commun 10, 1260 (2019).
29. Su, C. et al. 3D promoter architecture re-organization during iPSC-derived neuronal cell differentiation implicates target genes for neurodevelopmental disorders. Prog Neurobiol 102000 (2021) doi:10.1016/j.pneurobio.2021.102000.
30. Hammond, R. K. et al. Biological constraints on GWAS SNPs at suggestive significance thresholds reveal additional BMI loci. Elife 10, e62206 (2021).
31. Lasconi, C. et al. Variant-to-gene-mapping analyses reveal a role for the hypothalamus in genetic susceptibility to inflammatory bowel disease. Cell Mol Gastroenterology Hepatology (2020) doi:10.1016/j.jcmgh.2020.10.004.
32. Wagar, L. E. et al. Modeling human adaptive immune responses with tonsil organoids. Nature Medicine 27, 125-135 (2021)
33. Galani, I.-E. et al. Untuned antiviral immunity in COVID-19 revealed by temporal type I/III interferon patterns and flu comparison. Nat Immunol 22, 32-40 (2021).
34. Zhang, J.-Y. et al. Single-cell landscape of immunological responses in patients with COVID-19. Nat Immunol 21, 1107-1118 (2020).
35. Lan, X. et al. ZNF410 Uniquely Activates the NuRD Component CHD4 to Silence Fetal Hemoglobin Expression. Mol Cell 81, 239-254.e8 (2021).
36. Capoccia, B. J. et al. Transcription factor MIST1 in terminal differentiation of mouse and human plasma cells. Physiol Genomics 43, 174-186 (2011).
37. Liu, X. et al. Transcription factor achaete-scute homologue 2 initiates follicular T-helper-cell development. Nature 507, 513-516 (2014).
38. Sobreira, D. R., et al. Extensive pleiotropism and allelic heterogeneity mediate metabolic effects of IRX3 and IRX5. Science 372, 1085-1091 (2021).
39. Karlas, A., Machuy, N. & Meyer, T. F. Human host cell factors crucial for influenza virus replication identified by genome-wide RNAi screen. New Biotechnol 27, S84 (2010).
40. Sun, C.-T. et al. Transcription Repression of Human Hepatitis B Virus Genes by Negative Regulatory Element-binding Protein/SON*. J Biol Chem 276, 24059-24067 (2001).
41. Danchin, A. & Marliere, P. Cytosine drives evolution of SARS-CoV-2. Environ Microbiol 22, 1977-1985 (2020).
42. Luo, R., Wong, Y.-S. & Lam, T.-W. Tracking cytosine depletion in SARS-CoV-2. Biorxiv 2020.10.26.354787 (2020) doi:10.1101/2020.10.26.354787.
43. Sette, A. & Crotty, S. Adaptive immunity to SARS-CoV-2 and COVID-19. Cell (2021) doi:10.1016/j.cell.2021.01.007.
44. Yin, X. et al. MDA5 Governs the Innate Immune Response to SARS-CoV-2 in Lung Epithelial Cells. Cell Reports 34, 108628 (2021).
45. Yuen, C.-K. et al. SARS-CoV-2 nsp13, nsp14, nsp15 and orf6 function as potent interferon antagonists. Emerg Microbes Infec 9, 1-29 (2020).
46. Wu, J. et al. SARS-CoV-2 ORF9b Inhibits RIG-I-MAVS Antiviral Signaling by Interrupting K63-Linked Ubiquitination of NEMO. Cell Reports 34, 108761 (2021).
47. Lokugamage, K. G. et al. Type I Interferon Susceptibility Distinguishes SARS-CoV-2 from SARS-CoV. J Virol 94, (2020).
48. Blanco-Melo, D. et al. Imbalanced Host Response to SARS-CoV-2 Drives Development of COVID-19. Cell 181, 1036-1045.e9 (2020).
49. Bastard, P. et al. Autoantibodies against type I IFNs in patients with life-threatening COVID-19. Science 370, eabd4585 (2020).
50. Acharya, D., Liu, G. & Gack, M. U. Dysregulation of type I interferon responses in COVID-19. Nat Rev Immunol 20, 397-398 (2020).
51. Hadjadj, J. et al. Impaired type I interferon activity and inflammatory responses in severe COVID-19 patients. Science 369, 718-724 (2020).
52. Zhang, Q. et al. Inborn errors of type I IFN immunity in patients with life-threatening COVID-19. Science 370, eabd4570 (2020).
53. Yize Li, Renner, D. M., Comar, C. E., Whelan, J. N., Reyes, H. M. SARS-CoV-2 induces double-stranded RNA-mediated innate immune responses in respiratory epithelial-derived cells and cardiomyocytes. PNAS 118, e2022643118 (2021).
54. Bauernfried, S. and Hornung, V. DPP9 restrains NLRP1 activation. Nat. Struct. Mol. Biol. doi: 10.1038/s41594-021-00580-y (2021).
55. Magg, T. et al. Heterozygous OAS1 gain-of-function variants cause an autoinflammatory immunodeficiency. Sci. Immunol. 6, eabf9564 (2021).
56. Vujkovic, M. et al. Discovery of 318 new risk loci for type 2 diabetes and related vascular outcomes among 1.4 million participants in a multi-ancestry meta-analysis. Nat Genet 52, 680-691 (2020).
57. Fang, L., Karakiulakis, G. & Roth, M. Are patients with hypertension and diabetes mellitus at increased risk for COVID-19 infection? Lancet Respir Medicine 8, e21 (2020).
58. Fingerlin, T. E. et al. Genome-wide association study identifies multiple susceptibility loci for pulmonary fibrosis. Nat. Genetics 45, 613-620 (2013).
59. Aziz, M., Fatima, R. & Assaly, R. Elevated interleukin-6 and severe COVID-19: A meta-analysis. J Med Virol 92, 2283-2285 (2020).
60. Ye, Q., Wang, B. & Mao, J. Cytokine Storm in COVID-19 and Treatment. J Infection 80, 607-613 (2020).
61. Mehta, P. et al. COVID-19: consider cytokine storm syndromes and immunosuppression. Lancet 395, 1033-1034 (2020).
62. Han, H. et al. Profiling serum cytokines in COVID-19 patients reveals IL-6 and IL-10 are disease severity predictors. Emerg Microbes Infec 9, 1123-1130 (2020).
63. Minami, M. et al. Prostaglandin E Receptor Type 4-associated Protein Interacts Directly with NFkB1 and Attenuates Macrophage Activation. J. Biol. Chem. 283, 9692-9703 (2008).
64. Chen, P., Zhou, J., Li, J., Zhang, Q., Zuo, Q. TIPE1 suppresses osteosarcoma tumor growth by regulating macrophage infiltration. Clin Transl Oncol 21, 334-341 (2019).
65. Musskopf, M. K., de Mattos, E. P., Bergink, S., Kampinga, H. H. Hsp40/DNAJ Chaperones. eLS, DOI: 10.1002/9780470015902.a0027633 (2018).
66. Zhou, S. et al. Paxbpl controls a key checkpoint for cell growth and survival during early activation of quiescent muscle satellite cells. PNAS 118, e2021093118 (2021).
67. Berghorn, K. A. et al. Smad6 Represses Dlx3 Transcriptional Activity through Inhibition of DNA Binding. J. Biol. Chem. 281, 20357-20367 (2006)
68. Puleston, D. J., Villa, M. & Pearce, E. L. Ancillary Activity: Beyond Core Metabolism in Immune Cells. Cell Metab 26, 131-141 (2017).
69. Wagar, L. E. et al. Modeling human adaptive immune responses with tonsil organoids. Nature Medicine 27, 125-135 (2021)
70. Shi, L. et al. IL-1 Transcriptional Responses to Lipopolysaccharides Are Regulated by a Complex of RNA Binding Proteins. J Immunol Baltim Md 1950 204, 1334-1344 (2020).
71. Wingett, S. et al. HiCUP: pipeline for mapping and processing Hi-C data. F1000research 4, 1310 (2015).
72. Cairns, J. et al. CHiCAGO: robust detection of DNA looping interactions in Capture Hi-C data. Genome Biol 17, 127 (2016).
73. Coetzee, S. G., Coetzee, G. A. & Hazelett, D. J. motifbreakR: an R/Bioconductor package for predicting variant effects at transcription factor binding sites. Bioinformatics 31, 3847-3849 (2015).
74. Khan, A. et al. JASPAR 2018: update of the open-access database of transcription factor binding profiles and its web framework. Nucleic Acids Res 46, gkx1126-(2017).
75. Shannon, P. et al. Cytoscape: A Software Environment for Integrated Models of Biomolecular Interaction Networks. Genome Res 13, 2498-2504 (2003).
76. Bray, N. L., Pimentel, H., Melsted, P. & Pachter, L. Near-optimal probabilistic RNA-seq quantification. Nat Biotechnol 34, 525-527 (2016).

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for alleviating severe Covid 19 symptoms and other inflammatory disease symptoms or in a patient in need thereof, comprising;
a) identifying in a nucleic acid containing biological sample, at least one gene selected from GART, OAS1, OAS2, c21orf49, PaXBP1, SON, IL10RB, IFNAR1, DNAJC28, AP000295.9, DPP9, TNAaIP8L1 FEMA1A and DLX3, or variant thereof, which is indicative of the presence of, or altered risk for, severe Covid 19 or other inflammatory disease;
c) treating said patient with an effective amount of at least one therapeutic agent which targets said gene harboring said causal variant, thereby alleviating Covid 19 or other inflammatory disease symptoms.

2. The method of claim 1, wherein said other inflammatory disease is selected from inflammatory bowel disease, Myasthenia Gravis, ulcerative colitis, type I diabetes, lupus, celiac disease, allergy, eczema, Autoimmune encephalitis, and rheumatoid arthritis and the method further comprises administration of a steroid.

3. The method of claim 1, wherein said agent is an inhibitor that inhibits the expression or function of said gene.

4. The method of claim 1, wherein said agent is an agonist that increase the expression or function of said gene.

5. The method of claim 1, wherein sentinel and proxy SNPs implicating GWAS causal variants and genes identified through 3D epigenomics assays, are provided in FIG. 4B.

6. The method of claim 1, wherein identified genes and suitable therapeutic agents are listed in Table 4.

7. The method of claim 4, wherein said disease is severe Covid-19 and said gene is GART and the agent is a GART agonist.

8. A method for identifying an agent useful for the treatment of severe Covid 19 or other inflammatory disease, comprising;
a) incubating
i) a cell harboring at least one gene comprising an informative SNP for severe Covid 19 or other inflammatory disease in a cell type of interest and
ii) a cell which lacks said informative SNP in the presence and absence of an agent which modulates the function or expression of at least one gene target associated with one or more of severe Covid 19 symptoms and inflammatory disease symptoms; and
b) identifying agents which alter one or more of the inflammation modulating functions of said gene in cells harboring said SNP relative to those lacking said SNP.

9. The method of claim 8, wherein said cells are selected from tonsil follicular T helper cells, naïve CD4+ T cells, naïve CD8+ T cells, memory CD4+ T cells, memory CD8+ T cells, cytotoxic T lymphocytes, naïve B cells, germinal center B cells, Th1 cells, Th2 cells, Th17 cells, NK cells, dendritic cells, monocytes

10. The method of claim 8, wherein said gene is selected from GART, OAS1, OAS2, OAS3, C21orf49, PaXPB1, SON, IL10RB, IFNAR1, INFAR2, DNAJC28, TNFAIBPL1 AP000295.9, FEM1A, DPP9, and DLX3.

11. The method of claim 8, wherein said agent is provided in Table 4.

12. The method of claim 8, wherein said inflammatory disease is selected from inflammatory bowel disease, ulcerative colitis, Myasthenia Gravis, type I diabetes, lupus, celiac disease, allergy, eczema, autoimmune encephalitis, and rheumatoid arthritis.

13. A method for treatment of severe Covid 19 inflammatory disease and alleviating Covid symptoms as claimed in claim 1, in a patient in need thereof, comprising administration of an effective amount of a GART agonist, said treatment alleviating Covid 19 symptoms.

14. The method of claim 13, wherein said agent is a GART agonist comprising one or more of THF, fGAR and recombinantly produced GART.

15. The method of claim 13, further comprising administration of a steroid.

16. The method of claim 1, wherein said therapeutic agent is an antagonist or inhibitor.

17. The method of claim 16, wherein said gene is GART and said inhibitor is selected from lometrol, pemetrexed and pelitrexol.

18. The method of claim 13, further comprising administration of a modulator of one or more genes selected from OAS1, OAS2, OAS3, C21orf49, PaXPB1, SON, IL10RB, IFNAR1, INFAR2, DNAJC28, TNFAIBPL1 AP000295.9, FEM1A, DPP9, and DLX3.

19. A method for alleviating symptoms of an inflammatory disease selected from inflammatory bowel disease, ulcerative colitis, Myasthenia Gravis, type I diabetes, lupus, celiac disease, allergy, eczema, autoimmune encephalitis, and rheumatoid arthritis in a patient in need thereof, comprising;
a) identifying in a nucleic acid containing biological sample, at least one gene selected from OAS1, OAS2, OAS3, C21orf49, PaXPB1, SON, IL10RB, IFNAR1, INFAR2, DNAJC28, TNFAIBPL1 AP000295.9, FEM1A, DPP9, and DLX3, or variant thereof, which is indicative of the presence of, or altered risk for said inflammatory disease;
c) treating said patient with an effective amount of at least one therapeutic agent which targets said gene harboring said causal variant, thereby alleviating inflammatory disease symptoms.

20.-21. (canceled)

22. The method of claim 19, wherein said therapeutic agent is comprises a nucleic acid which modulates expression of at least one gene and optionally a steroid.
